(19) United States
(12) Patent Application Publication
(10) Pub. No.: US 2007/0031902 A1
(43) Pub. Date: Feb. 8, 2007

(54) Predictive Methods For Cancer Chemotherapy

(76) Inventors: Gary Anthony Pestano, Oro Valley, AZ (US); Linda Kay Samadzadeh, Tacoma, WA (US); Kristie Ann Vanpatten, Oro Valley, AZ (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 11/462,356

(22) Filed: Aug. 3, 2006

Related U.S. Application Data
(60) Provisional application No. 60/705,805, filed on Aug. 3, 2005.

(51) Int. Cl. G01N 33/574

(52) U.S. Cl. 435/7.23

(57) ABSTRACT

This invention provides methods and reagents for determining or predicting response to cancer therapy, as well as dual therapy treatments.

This application claims priority to U.S. provisional application Ser. no. 60/705,805, filed Aug. 3, 2005.

### BACKGROUND OF THE INVENTION

1. Field of the Invention

This invention relates to methods and reagents for determining or predicting response to cancer therapy in an individual. The invention also relates to methods for using image analysis of immunohistochemically-stained samples to quantify gene expression, phosphorylation, or both for genes of cancer-related metabolic pathways, including mTOR, HIF-1α, pERK, and/or pMEK expression and phosphorylation (activation). The invention also relates to dual therapeutic treatments directed to a plurality of said cancer-related metabolic pathways.

2. Background of the Invention

A primary goal of cancer therapy is to selectively kill or inhibit uncontrolled growth of malignant cells while not adversely affecting normal cells. Traditional chemotherapeutic drugs are highly cytotoxic agents that preferably have greater affinity for malignant cells than for normal cells, or at least preferentially affect malignant cells based on their high rate of cell growth and metabolic activity. However, these agents often harm normal cells. Cancer treatment therapies that can target the malignant cells and spare the normal cells, referred to as targeted therapies, are part of a new wave of chemotherapeutics. Such new therapies are important for solid tumor cancers, which continue to be viewed as chronic conditions, creating a need for long-term treatments with less side-effects, as well as methods for developing, assessing, and predicting success for such therapies.

Targeted cancer therapies attempt to block the growth and spread of cancer cells by interfering with molecules or intracellular pathways that are specific to the tumor cell and carcinogenesis, in contrast with conventional chemotherapeutic or chemopreventive agents that are used to produce growth arrest, terminal differentiation and cell death in dividing cells. While these conventional treatment modalities preferentially affect cancerous or precancerous cells, their intrinsic non-specificity deleteriously affects normal cells as well.

Traditional therapeutic cancer regimens have been developed based upon results from large scale trials and rely upon predictive outcomes for a wide variety of patients and tumors. The capacity to tailor therapies to the individual patient and tumor may provide more efficacious treatments for malignancies with fewer side-effects. Furthermore, the ability to monitor the progression of the cancer treatment and adjust the therapy accordingly would allow for a more rapid reaction to individual differences in response to therapeutic regimens that have been previously developed using data from a wide group of patients.

The finding that the growth of solid tumors and hematologic malignancies are dependent on angiogenesis has suggested that one mechanism to combat tumor growth is to inhibit pathways involved in the development of nascent blood vessels (Folkman, 2003, Curr. Mol. Med. 3:643-51; Folkman, 1971, New England Journal of Medicine, 285:1182-86). In addition, this observation has been extended to a complementary strategy, i.e., to examine the role of oxygen in the transformed cell itself to determine possible therapeutic routes.

Several signaling pathways have emerged as important targets for the understanding and treatment of oncogenesis; however, diversity of ligands and receptors and resulting outcomes from receptor signaling have, in part, contributed to the difficulties in identifying robust diagnostic candidate biomarkers for targeted therapies Nevertheless, signaling pathways showing promise as targets include growth factor and nutrient responsive signal transduction pathways. The growth factor and nutrient pathways regulate cell growth and metabolism in response to intracellular and environmental cues. These signaling pathways are often altered or dysregulated in cancer resulting in a phenotype of uncontrolled growth and invasion of surrounding tissue. The growth factor or epidermal growth factor (“EGF”) pathway and the nutrient mammalian target of rapamycin (mTOR) pathway are both targets of active research in cancer diagnosis and treatment.

EGF is a growth factor that activates protein-receptor tyrosine kinase (“RTK”) activity to initiate a signal transduction cascade resulting in changes in cell growth, proliferation and differentiation. EGF and its downstream targets, including ras/raf, mek, and erk, have been shown to be involved in the pathogenesis and progression of several different cancers. This pathway and its signaling molecules provide attractive targets for therapeutic intervention and such approaches are in development (Stadler, 2005, Cancer, 104(11):2323-33; Normanno, et al., 2006, Gene, 366(1):2-16). Agents that target EGF and its receptor include bevacizumab, PTK787, SU011248 and BAY 43-9006. The BAY 43-9006 compound has also been shown to inhibit the downstream targets in the EGF pathway including raf, mek and erk (Stadler, 2005 Cancer, 104(11):2323-33).

The nutrient responsive signaling pathways, including the mTOR pathway, are also critical in oncogenesis, particularly solid tumor and hematological malignancies. mTOR is a serine/threonine kinase responsible for cell proliferation/survival signaling by inducing cell-cycle progression from G1 to S phase in response to nutrient availability, (Maloney and Rees, 2005, Reproduction, 130:401-410). Dysregulation in the mTOR signaling pathway has been linked to oncogenesis. Like the EGF pathway, the mTOR pathway includes multiple small molecule targets for therapeutic intervention. mTOR inhibitors have been developed including rapamycin and its analogues CCI-779, RAD001, and AP23573. Such treatments are currently in phase II-III clinical trials (Janus, et al., 2005, Cell Mol Biol Lett, 10(3):479-98).

The intersection of RTK or EGF signaling and the mTOR-driven pathways has garnered significant interest in the development of targeted therapies. Specific targets include modulation of the EGF family and of the mTOR/HIF-1α pathway. Both pathways promote solid tumor growth and hematological malignancies.

HIF-1α (hypoxia-inducible factor-1), a downstream target in the mTOR pathway, is a dimeric transcription factor involved in oxygen homeostasis in mammalian cells. The complex is composed of α and β subunits which, upon reduced oxygen availability, bind to an enhancer known as the hypoxia-response element (HRE). Hypoxia-response elements were found to be involved in the regulation of genes such as erythropoietin, Vascular Endothelial Growth Factor (VEGF), and Flt-1 (a VEGF receptor). In addition to controlling expression of genes involved in vascularization and erythropoiesis, HIF-1α mediates transcription of genes with protein products involved in proliferation, survival, and metabolism (Semenza, 2000, Journal ofApplied Physiology, 88:1474-80).

Stabilization of HIF-1α also has been shown to be downstream of several signaling cascades under normoxia, including those activated by insulin, EGF, FGF, and TNF-α. It is likely that HIF-1α is regulated in these systems through a common signaling component. In fact, both the P13K/Akt/mTor and ras/raf/mek-1/(erk½) pathways have been implicated in HIF-1α function in a number of cell types (Powis and Kirkpatrick, 2004, Molecular Cancer Therapeutics, 3:647-54). These results provide additional evidence that there may be multiple events, such as downstream HER-2 signaling or hypoxia, which are necessary but not sufficient for controlling HIF-1α expression.

While studies have examined the role of both the EGF and mTOR pathways in the control and treatment of cancer, there is little known about the interaction between these two pathways via HIF1α and the possible consequences of therapeutic intervention in one pathway or the other. Such connections could have significant impact on the diagnosis and treatment of malignancies. Furthermore, HIF-1α is expressed in the majority of solid tumors examined (Ryan, et al., 1998, EMBO, 17:3005-15; Powis and Kirkpatrick, 2004, Molecular Cancer Therapeutics, 3:647-54). As a clinical marker, a high level of HIF-1α expression is correlated with a poor prognosis in lymph-node negative breast cancer, oropharyngeal carcinoma, early cervical carcinoma, oligodendrogliomas, and non-small cell lung carcinomas (Bos et al., 2003, Cancer, 97:1573-81).

There exists a need in the art to develop diagnostic biomarkers to allow for the screening and rapid detection of changes in various intracellular signaling molecules during cancer treatment in order to monitor the effects of treatment directed against the mTOR pathway. There is also a need for improved identification methods of dual inhibitors towards the EGF and mTOR pathways.

### SUMMARY OF THE INVENTION

This invention provides reagents and methods for identifying and detecting expression or activation of biological markers of tumorigenesis in cells and tissue samples from cancer patients. The methods provided herein are useful for predicting or assessing a response (or predicting or assessing a lack of response) of an individual cancer patient to a particular treatment regimen.

In a first aspect, the invention provides methods for identifying a mammalian tumor that can be treated with a dual mTOR pathway inhibitor and EGF pathway inhibitor therapy, comprising the step of assaying a sample obtained from the mammalian tumor to detect a pattern of expression, phosphorylation or both expression and phosphorylation of a panel of two or more polypeptides consisting of:

(a) at least one polypeptide of the EGF pathway, and

(b) at least one polypeptide of the mTOR pathway

wherein the expression, phosphorylation or both expression and phosphorylation identifies mammalian tumors in need of dual mTOR pathway inhibitor and EGF pathway inhibitor therapy. The pattern of expression, phosphorylation or both expression and phosphorylation can be as compared to the pattern of expression, phosphorylation or both expression and phosphorylation of a non-tumor sample.

In certain embodiments, the at least one polypeptide of the EGF pathway comprises the phosphorylated ERK polypeptide; the phosphorylated MEK polypeptide, or both the phosphorylated ERK polypeptide and the phosphorylated MEK polypeptide. In other embodiments, the at least one polypeptide of the mTOR pathway comprises HIF-1α polypeptide, mTOR polypeptide, or both HIF-1α and mTOR polypeptide. In certain embodiments, the mammalian tumor is identified as treatable with a dual mTOR pathway inhibitor and EGF pathway inhibitor therapy when the detected pattern of expression, phosphorylation or both expression and phosphorylation of the panel of polypeptides in the sample is greater that the level of expression, phosphorylation or both expression and phosphorylation of the panel of polypeptides in a non-tumor control.

In yet further embodiments, both the at least one polypeptide of the EGF pathway comprises the phosphorylated ERK polypeptide; the phosphorylated MEK polypeptide, or both the phosphorylated ERK polypeptide and the phosphorylated MEK polypeptide; and the at least one polypeptide of the mTOR pathway comprises HIF-1α polypeptide, mTOR polypeptide, or both HIF-1α and mTOR polypeptide. In another embodiment of the invention, the at least one polypeptide of the EGF pathway comprises the phosphorylated ERK. polypeptide; and wherein the at least one polypeptide of the mTOR pathway comprises HIF-1α polypeptide. In yet other embodiments, the at least one polypeptide of the EGF pathway comprises the phosphorylated MEK polypeptide; and wherein the at least one polypeptide of the mTOR pathway comprises HIF-1α polypeptide. In certain embodiments, the mammalian tumor is identified as treatable with a dual mTOR pathway inhibitor and EGF pathway inhibitor therapy when the detected pattern of expression, phosphorylation or both expression and phosphorylation of the panel of polypeptides in the sample is greater that the level of expression, phosphorylation or both expression and phosphorylation of the panel of polypeptides in a non-tumor control.

In a second aspect, the invention provides methods for assessing a positive response to receiving a dual mTOR pathway inhibitor and EGF pathway inhibitor therapy in an individual, comprising
(a) obtaining a first tissue or cell sample from the individual before exposing the individual to the dual mTOR pathway inhibitor and EGF pathway inhibitor therapy;
(b) obtaining a second tissue or cell sample from the individual after exposing the individual to the dual mTOR pathway inhibitor and EGF pathway inhibitor therapy;
(c) detecting a pattern of expression, phosphorylation or both expression and phosphorylation of a panel of two or more polypeptides consisting of:
   (i) at least one polypeptide of the EGF pathway, and
   (ii) at least one polypeptide of the mTOR pathway in said first tissue or cell sample and said second tissue or cell sample;
(d) detecting a difference in the pattern of expression, phosphorylation or both expression and phosphorylation between the first tissue or cell sample and the second tissue or cell sample,
   wherein decreased expression, phosphorylation or both expression and phosphorylation between the second tissue or cell sample and the first tissue or cell sample shows a positive response to receiving the dual mTOR pathway inhibitor and EGF pathway inhibitor therapy

In certain embodiments, the at least one polypeptide of the EGF pathway comprises the phosphorylated ERK polypeptide; the phosphorylated MEK polypeptide, or both the phosphorylated ERK polypeptide and the phosphorylated MEK polypeptide. In other embodiments, the at least one polypeptide of the mTOR pathway comprises HIF-1α polypeptide, mTOR polypeptide, or both HIF-1α arid mTOR polypeptide. In yet other embodiments, both the at least one polypeptide of the EGF pathway comprises the phosphorylated ERK polypeptide; the phosphorylated MEK polypeptide, or both the phosphorylated ERK polypeptide and the phosphorylated MEK polypeptide; and the at least one polypeptide of the mTOR pathway comprises HIF-1α polypeptide, mTOR polypeptide, or both HIF-1α and mTOR polypeptide.

In yet other embodiments, the at least one polypeptide of the EGF pathway comprises the phosphorylated ERK polypeptide; and the at least one polypeptide of the mTOR pathway comprises HIF-1α polypeptide. In further embodiments, the at least one polypeptide of the EGF pathway comprises the phosphorylated MEK polypeptide; and the at least one polypeptide of the mTOR pathway comprises HIF-1α polypeptide.

In a third aspect, the invention provides kits for identifying a mammalian tumor that can be treated with or assessing a positive response in an individual to receiving a dual mTOR pathway inhibitor and EGF pathway inhibitor therapy comprising at least two reagents for detecting the expression, phosphorylation or both expression and phosphorylation of polypeptides in the EGF pathway, the mTOR pathway, or both the EGF pathway and the mTOR pathway.

In certain embodiments, the at least two reagents detect the expression, phosphorylation or both expression and phosphorylation of a panel of polypeptides consisting of:

(a) at least one polypeptide of the EGF pathway, and

(b) at least one polypeptide of the mTOR pathway.

In other embodiments, the at least one polypeptide of the EGF pathway can be the phosphorylated form of ERK. In yet others, the at least one polypeptide of the EGF pathway can be the phosphorylated form of MEK. In other embodiments, the at least one polypeptide of the mTOR pathway can be HIF-1α. In yet others, the at least one polypeptide of the mTOR pathway can be mTOR.

In certain embodiments, the at least two reagents are antibodies. In yet others, the two reagents comprise: (a) at least one antibody that binds to an epitope of a polypeptide of the EGF pathway, and (b) at least one antibody that binds to an epitope of a polypeptide of the mTOR pathway. In yet other embodiments, the kit contains a detection reagent. In other embodiments, the at least one antibody that binds to an epitope of a polypeptide of the EGF pathway binds to an epitope of ERK, or the phosphorylated form of ERK. In another embodiment, it binds to an epitope of MEK, or the phosphorylated form of MEK. In yet another embodiment, the at least one antibody that binds to an epitope of a polypeptide of the mTOR pathway binds to an epitope of HIF-1α. In yet another embodiment, it binds to an epitope of mTOR.

In a fourth aspect, the invention provides therapeutic treatments comprising an inhibitor of the EGF pathway and an inhibitor of the mTOR pathway, such as HIF-1α. In further embodiments, the inhibitor of the EGF pathway is either an inhibitor of MEK phosphorylation or an inhibitor of ERK phosphorylation. In yet further embodiments, the inhibitor of the mTOR pathway is an inhibitor of mTOR. In further embodiments, the inhibitor is rapamycin. In certain embodiments, the inhibitor of the mTOR pathway is an inhibitor of HIF-1α. In yet further embodiments, the inhibitor is PX-478.

In a fifth aspect, the invention provides method for identifying a mammalian tumor that can be treated with a dual mTOR pathway inhibitor and EGF pathway inhibitor therapy, comprising the step of assaying a mammalian tumor sample obtained from an individual that has received an mTOR pathway inhibitor to detect a pattern of expression, phosphorylation or both expression and phosphorylation of at least one polypeptide of the EGF pathway; wherein the expression, phosphorylation or both expression and phosphorylation of the at least one polypeptide of the EGF pathway identifies the mammalian tumor as treatable with a dual mTOR pathway inhibitor and EGF pathway inhibitor therapy.

In certain embodiments, the at least one polypeptide of the EGF pathway comprises a phosphorylated ERK polypeptide; a phosphorylated MEK polypeptide, or both a phosphorylated ERK polypeptide and a phosphorylated MEK polypeptide. In other embodiments, the detected pattern of expression, phosphorylation or both expression and phosphorylation of the at least one polypeptide of the EGF pathway is compared to a pattern of expression, phosphorylation or both expression and phosphorylation of the at least one polypeptide of the EGF pathway in a control; wherein an increased levels of the at least one polypeptide of the EGF pathway in the sample as compared to the levels of the panel of polypeptides in the control identifies the mammalian tumor as treatable with a dual mTOR pathway inhibitor and EGF pathway inhibitor therapy. The control can be a tumor sample from the individual before the individual received the mTOR pathway inhibitor.

In a sixth aspect, the invention provides methods for identifying a mammalian tumor that can be treated with a dual mTOR pathway inhibitor and EGF pathway inhibitor therapy, comprising the steps of (1) treating a mammalian tumor sample obtained from an individual with an mTOR pathway inhibitor; and (2) detecting a pattern of expression, phosphorylation or both expression and phosphorylation of at least one polypeptide of the EGF pathway; wherein the expression, phosphorylation or both expression and phosphorylation of the at least one polypeptide of the EGF pathway identifies the mammalian tumor as treatable with a dual mTOR pathway inhibitor and EGF pathway inhibitor therapy.

In certain embodiments, the at least one polypeptide of the EGF pathway comprises a phosphorylated ERK polypeptide; a phosphorylated MEK polypeptide, or both a phosphorylated ERK polypeptide and a phosphorylated MEK polypeptide. In other embodiments, the detected pattern of expression, phosphorylation or both expression and phosphorylation of the at least one polypeptide of the EGF pathway is compared to a pattern of expression, phosphorylation or both expression and phosphorylation of the at least one polypeptide of the EGF pathway in a control; wherein an increased levels of the at least one polypeptide of the EGF pathway in the sample as compared to the levels of the panel of polypeptides in the control identifies the mammalian tumor as treatable with a dual mTOR pathway inhibitor and EGF pathway inhibitor therapy. The control can be a tumor sample from the individual that did not receive the mTOR pathway inhibitor.

Specific embodiments of the present invention will become evident from tile following more detailed description of certain preferred embodiments and the claims.

### DESCRIPTION OF THE DRAWINGS

FIGS. 1A-H illustrates representative images of pathway biomarker staining in Jurkat cells (PAKT, mTOR, pmTOR, pTSC2, HIF-1α, pMEK, pS6, and p4EBP, respectively). Both Control and DFO-treated conditions are shown, with a magnification of 40×.

FIG. 2 shows a schematic diagram of cell signaling after induction of HIF-1α. The relative expression of biomarkers in the RTK and mTOR pathways are shown after 1HC and image analysis.

FIG. 3 illustrates the expression of HIF-1α as evaluated in Jurkat (FIG. 3A) and HT1080 (FIG. 3B) cell lines under normoxic and hypoxic (desferrioxamine (“DFO”)) conditions. The cell lines were reviewed by a pathologist, and the magnification is indicated.

FIG. 4A are representative immunohistochemistry (IHC) images of HT1080 cells treated with DFO and various concentrations of the HIF-1α inhibitor PX-478 (40×) (Control, DFO alone, DFO+25 μM PX-478, DFO+50 μM PX-478, and DFO+75 μM PX-478). FIG. 4B shows the image analysis that was performed on the IHC images.

FIG. 5A shows Western Blot analysis of HT1080 cells treated with a small molecule HIF-1α inhibitor PX-478. The lanes include: (1) vehicle-treated; (2) DFO only; (3) DFO+25 μM drug; (4) DFO+50 μM drug; and (5) DFO+75 μM drug. FIG. 5B shows a Western Bolt with Laminin detection to determine equivalent loading. Figure SC shows the results of densitometry of the Western Blots to quantify HIF-1α expression in the presence of the inhibitor.

FIG. 6 illustrates the fluorescence-activated cell sorting (FACS) results of hypoxia-induced HIF-1α expression in a Jurkat tumor cell line.

FIG. 7A is a representative image of pMEK staining in HT1080 cells treated with DFO and HIF-1α inhibitor (Control, DFO alone, DFO+25 μM PX-478, DFO+50 μM PX-478, and DFO+75 μM PX-478). FIG. 7B shows the present inhibition of expression of HER/mTOR Pathway Markers (pS6, pAKT, and pMEK and pERK) in HT1080 cells. The cells were analyzed after treatment with DFO and various concentrations of the HIF-1α inhibitor PX-478. Dividing cells were analyzed for pMEK expression. The data represents the percent inhibition of hypoxic effects (DFO-treatment) on the respective marker calculated according to the following formula: (100-((% treated÷%baseline)×100)). FIG. 7C illustrates cells analyzed for pMEK expression in HT1080 cells after treatment with DFO and various concentrations of the HIF-1α inhibitor. The data are shown for hypoxia and the inhibition of hypoxic effects (DFO treatment±HIF-1α inhibitor) on the pMEK biomarker.

FIG. 8 shows representative images of Dual Brightfield IHC double labeling for (A) HIF-1α (Fast Red detection reagent) and pMEK (DAB-brown detection reagent) counter-stain (hemotoxylin) and (B) HIF-1α (DAB-brown detection reagent) and pMEK (NBT/BCIP-Blue detection reagent) counter-stain (Nuclear Fast Red). The respective biomarkers are labeled in FIGS. 8A-B.

FIG. 9A-B shows representative images of QDOT Fluorescent IHC double labeling for HIF-1α and (A) pMEK or (B) pERK. The HIF-1α expression cells are found mainly in the hypoxic-labeled field in FIG. 9, while pMEK and pERK are mainly found in the proliferative-labeled field. FIG. 9C shows the results of simultaneous detection of HIF-1α and pMEK with QDOT Fluorescent IHC double labeling, including an image with selected cells from the field.

FIG. 10 is a schematic of cross talk between EGF and MTOR pathways.

FIG. 11 illustrates representative images of HIF-1α protein detection using different detection kits and IHC.

FIG. 12 is a schematic diagram showing the assay development Matrix (CLAD™).

FIG. 13 is a schematic diagram showing HIF-1α Mouse Monoclonal Assay development.

### DETAILED DESCRIPTION OF THE PREFERRED EMBODIMENTS

This invention provides methods for predicting response in cancer subjects to cancer therapy, including cancer patients. In addition, this invention provides predictive biomarkers to identify those cancer patients for whom administering a therapeutic agent will be most effective, including a dual inhibitor therapy. Specifically, this invention provides predictive biomarkers for assessing or monitoring the efficacy of dual therapeutic agents targeted to members of the EGF pathway or the mTOR pathway. Moreover, this invention provides kits for identifying a mammalian tumor in need of or assessing a response in a subject to receiving a dual mTOR pathway inhibitor. Furthermore, this invention provides a dual inhibitor therapeutic treatment.

In contrast to traditional anticancer methods, where chemotherapeutic drug treatment is undertaken as an adjunct to and after surgical intervention, neoadjuvant (or primary) chemotherapy consists of administering drugs as an initial treatment in certain cancer patients. One advantage of such an approach is that, for primary tumors of more than 3 cm, it permits the later or concomitant use of conservative surgical procedures (as opposed to, e.g., radical mastectomy in breast cancer patients) for the majority of patients, due to the tumor shrinking effect of the chemotherapy. Another advantage is that for many cancers, a partial and/or complete response is achieved in about two-thirds of all patients. Finally, because the majority of patients are responsive after two to three cycles of chemotherapeutic treatment, it is possible to monitor the in vivo efficacy of the chemotherapeutic regimen employed, in order to identify patients whose tumors are non-responsive to chemotherapeutic treatment. Timely identification of non-responsive tumors allows the clinician to limit a cancer patient's exposure to unnecessary side-effects of treatment and to institute alternative treatments. Unfortunately, methods present in the art, including histological examination, are insufficient for optimum application of such timely and accurate identification. The present invention provides methods for developing more informed and effective regimes of therapy that can be administered to cancer patients with an increased likelihood of an effective outcome (i.e., reduction or elimination of the tumor).

A cancer diagnosis, both an initial diagnosis of disease and subsequent monitoring of the disease course (before, during, or after treatment) is conventionally confirmed through histological examination of cell or tissue samples removed from a patient. Clinical pathologists need to be able to accurately determine whether such samples are benign or malignant and to classify the aggressiveness of tumor samples deemed to be malignant, because these determinations often form the basis for selecting a suitable course of patient treatment. Similarly, the pathologist needs to be able to detect the extent to which a cancer has grown or gone into remission, particularly as a result of or consequent to treatment, most particularly treatment with chemotherapeutic or biological agents.

Histological examination traditionally entails tissue-staining procedures that permit morphological features of a sample to be readily observed under a light microscope. A pathologist, after examining the stained sample, typically makes a qualitative determination of whether the tumor sample is malignant. It is difficult, however, to ascertain a tumor's aggressiveness merely through histological examination of the sample, because a tumor's aggressiveness is often a result of the biochemistry of the cells within the tumor, such as protein expression or suppression and protein phosphorylation, which may or may not be reflected by the morphology of the sample. Therefore, it is important to be able to assess the biochemistry of the cells within a tumor sample. Further, it is desirable to be able to observe and quantitate both gene expression and protein phosphorylation of tumor-related genes or proteins, or more specifically cellular components of tumor-related signaling pathways.

Cancer therapy can be based on molecular profiling of tumors rather than simply their histology or site of the disease. Elucidating the biological effects of targeted therapies in tumor tissue and correlating these effects with clinical response helps identify the predominant growth and survival pathways operative in tumors, thereby establishing a pattern of likely responders and conversely providing a rational for designing strategies to overcome resistance. For example, successful diagnostic targeting of a growth factor receptor must determine if tumor growth or survival is being driven by the targeted receptor or receptor family, by other receptors not targeted by the therapy, and whether downstream signaling suggests that another oncogenic pathway is involved. Furthermore, where more than one signaling pathway is implicated, members of those signaling pathways can be used as diagnostic targets to determine if a dual inhibitor therapy will be or is effective.

In order for chemotherapy to be effective, the medications should destroy tumor cells and spare the normal body cells, particularly those normal cells that may be adjacent or in proximity to the tumor. This can be accomplished, inter alia, by using medications that affect cell activities that go on predominantly in cancer cells but not in normal cells. One difference between normal and tumor cells is the amount of oxygen in the cells; many tumor cells are oxygen deficient and are “hypoxic.” Mammalian cells have an array of responses that maintain oxygen homeostasis that exists as a balance between the requirement for oxygen as an energy substrate and the inherent risk of oxidative damage to cellular macromolecules. The molecular basis for a variety of cellular and systemic mechanisms of oxygen homeostasis are now being identified and the mechanisms have been found to occur at every regulatory level, including gene transcription, protein translation, posttranslational modification, and cellular localization (Harris, 2002, Nat Rev. 2:38-47).

Hypoxic cancer cells occur for a number of reasons. Oxygen is only able to diffuse 100-180 microns from a capillary to cells before it is completely metabolized. Therefore, any cell located greater than this distance from a blood vessel will be hypoxic. Hypoxia may occur when aberrant blood vessels are shut down by becoming compressed or obstructed by growth, a feature commonly observed during the rapid growth of tumors. Cells that become hypoxic convert to a glycolytic metabolism, become resistant to apoptosis (programmed cell death), and are more likely to migrate to less hypoxic areas of the body (metastasis). Hypoxic cells also produce pro-angiogenic factors, such as vascular endothelial growth factor (VEGF), which stimulate new blood vessel formation from existing vasculature, increasing tumor oxygenation and, ultimately, tumor growth. For this reason, hypoxic tumors are the most pro-angiogenic and aggressive of tumors.

Automated (computer-aided) image analysis systems known in the art can augment visual examination of tumor samples. In a representative embodiment, the cell or tissue sample is exposed to detectably-labeled reagents specific for a particular biological marker, and the magnified image of the cell is then processed by a computer that receives the image from a charge-coupled device (CCD) or camera such as a television camera. Such a system can be used, for example, to detect and measure expression and activation levels of HIF-1α, pMEK, pERK, mTOR, pmTOR, pAKT, pTSC2, pS6, and p4EBP1 in a sample, or any additional diagnostic biomarkers. Thus, the methods of the invention provide more accurate cancer diagnosis and better characterization of gene expression in histologically identified cancer cells, most particularly with regard to expression of tumor marker genes or genes known to be expressed in particular cancer types and subtypes (e.g., having different degrees of malignancy). This information permits a more informed and effective regimen of therapy to be administered, because drugs with clinical efficacy for certain tumor types or subtypes can be administered to patients whose cells are so identified.

Another drawback of conventional anticancer therapies is that the efficacy of specific chemotherapeutic agents in treating a particular cancer in an individual human patient is unpredictable. In view of this unpredictability, the art is unable to determine, prior to starting therapy, whether one or more selected agents would be active as anti-tumor agents or to render an accurate prognosis or course of treatment in an individual patient. This is especially important because a particular clinical cancer may present the clinician with a choice of treatment regimens, without any current way of assessing which regimen will be most efficacious for a particular individual. It is an advantage of the methods of this invention that they are able to better assess the expected efficacy of a proposed therapeutic agent (or combination of agents) in an individual patient. The claimed methods are advantageous for the additional reasons that they are both time- and cost-effective in assessing the efficacy of chemotherapeutic regimens and are minimally traumatic to cancer patients.

Methods of this invention can be used to identify a mammalian tumor that responds to either an mTOR pathway inhibitor, or a dual mTOR pathway inhibitor and EGF pathway inhibitor therapy. Further, methods of this invention can be used to select a subject with cancer for a dual treatment with a molecule targeting a member of the mTOR pathway and the EGF pathway. Moreover, methods of this invention can be used to identify a mammalian tumor that does not respond to mTOR-directed therapies. Further, methods of this invention can be used to select a subject with cancer to not receive treatment with a molecule targeting member of the mTOR pathway, including MTOR and HIF- 1α.

By “mTOR pathway inhibitor” is meant an inhibitor or the expression or activation, or both expression or activation, of a member of the mTOR pathway. For example, an mTOR pathway inhibitor can inhibit the expression or activation, or both, of AKT, mTOR, pTSC2, HIF-1α, pS6, p4EBP1, pI3K, STAT3, as well as any receptor or receptor ligand that activates any component of the mTOR pathway. This list of member of the mTOR pathway is exemplary, and is not meant to be exhaustive.

By “EGF pathway inhibitor” is meant an inhibitor or the expression or activation, or both expression or activation, of a member of the EGF pathway. For example, an EGF pathway inhibitor can inhibit the expression or activation, or both, of RAS/RAF, MEK, and ERK, as well as any receptor, such as HER1, HER2, HER3, or HER4 or receptor ligand, such as EGF, TGF-A, Epiregulin, NRG1-4, or Growth Factor, that activates any component of the EGF pathway. This list of member of the EGF pathway is exemplary, and is not meant to be exhaustive.

For subjects considered for treatment with an mTOR pathway inhibitor, specifically an mTOR or HIF-1α inhibitor, it is necessary to consider additional biomarkers beyond the presence of the target mTOR or HIF-1α, at least because the status of components of the EGF pathway, specifically pERK and pMEK, affect mTOR pathway inhibitor therapy response in cancer patients. Therefore, HIF-1α expression alone does not necessarily predict overall response to mTOR pathway inhibitors.

Before administration of an mTOR pathway-targeted therapy, a panel of diagnostics of each tumor is used according to the methods of this invention to find the best candidate for each therapy. According to the methods of this invention, treatment by an mTOR pathway-targeted therapy, such as rapamycin or PX-478, may not be effective unless an EGF pathway inhibitor is used in combination. For example, where there are high levels of expression of components of the EGF pathway, such as pERK and pMEK, an mTOR pathway-targeted therapy is not effective. Use of the methods of this invention permits a clinician to choose a more effective combination of targeted therapies for cancer patients.

The mTOR pathway therapies of the present invention can include, for example, rapamycin and its analogues CCI-779, RAD001, and AP23573, as well as inhibitors of HIF-1α. Further, the EGF pathway inhibitors can include, for example, bevacizumab, PTK787, SUO 1248 and BAY 43-9006.

Patterns of expression and phosphorylation of polypeptides are detected and quantified using methods of the present invention. More particularly, patterns of expression and phosphorylation of polypeptides that are cellular components of a tumor-related signaling pathway are detected and quantified using methods of the present invention. For example, the patterns of expression and phosphorylation of polypeptides can be detected using biodetection reagents specific for the polypeptides, including but not limited to antibodies. Alternatively, the biodetection reagents can be nucleic acid probes.

As used with the inventive methods disclosed herein, a nucleic acid probe is defined to be a collection of one or more nucleic acid fragments whose hybridization to a sample can be detected. The probe may be unlabeled or labeled so that its binding to the target or sample can be detected. The probe is produced from a source of nucleic acids from one or more particular (preselected) portions of the genome, e.g., one or more clones, an isolated whole chromosome or chromosome fragment, or a collection of polymerase chain reaction (PCR) amplification products. The nucleic acid probe may also be isolated nucleic acids immobilized on a solid surface (e.g., nitrocellulose, glass, quartz, fused silica slides), as in an array. The probe may be a member of an array of nucleic acids as described, for instance, in WO 96/17958. Techniques capable of producing high density arrays can also be used for this purpose (see, e.g., Fodor (1991) Science 767-773; Johnston (1998) Curr. Biol. 8: R171-R174; Schummer (1997) Biotechniques 23: 1087-1092; Kern (1997) Biotechniques 23: 120-124; U.S. Pat. No. 5,143,854). One of skill will recognize that the precise sequence of the particular probes can be modified to a certain degree to produce probes that are “substantially identical,” but retain the ability to specifically bind to (i.e., hybridize specifically to) the same targets or samples as the probe from which they were derived. The term “nucleic acid” refers to a deoxyribonucleotide or ribonucleotide in either single- or double-stranded form. The term encompasses nucleic acids, i.e., oligonucleotides, containing known analogues of natural micleotides that have similar or improved binding properties, for the purposes desired, as the reference nucleic acid. The term also includes nucleic acids which are metabolized in a manner similar to naturally occurring nucleotides or at rates that are improved for the purposes desired. The term also encompasses nucleic-acid-like structures with synthetic backbones. One of skill in the art would recognize how to use a nucleic acid probes for screening of cancer cells in a sample by reference, for example, to U.S. Pat. No. 6,326,148, directed to screening of colon carcinoma cells.

Polypeptides associated with cancer can be quantified by image analysis using a suitable primary antibody against biomarkers, including but not limited HIF-1α, pMEK, pERK, mTOR, pmTOR, pAKT, pTSC2, pS6, and p4EBP 1, detected directly or using an appropriate secondary antibody (such as rabbit anti-mouse IgG when using mouse primary antibodies) and/or a tertiary avidin (or Strepavidin) biotin complex (“ABC”).

Examples of reagents useful in the practice of the methods of the invention as exemplified herein include antibodies specific for HIF-1α, including but not limited to the mouse monoclonal antibody VMSI 760-4285, obtained from Ventana Medical Systems, Inc. (Tucson, Ariz.). Other reagents useful in the practice of the methods of this invention include, but are not limited to, rabbit polyclonal antibody Abcam 2732 specific to mTOR, rabbit polyclonal antibody CST 2971 specific to pmTOR, rabbit polyclonal antibody CST 3614 specific to mTSC2, rabbit polyclonal antibody CST 2211 specific to pS6, rabbit monoclonal antibody CST 3787 specific to pAKT, rabbit polyclonal antibody CST 9121 specific to pMEK, rabbit polyclonal antibody VMSI 760-4228 specific to mERK (p44/p42), and rabbit polyclonal antibody CST 9455 specific to m4EBP1.

Further, the pattern of expression, phosphorylation, or both expression and phosphorylation of the predictive polypeptides can be compared to a non-tumor tissue or cell sample. The non-tumor tissue or cell sample can be obtained from a non-tumor tissue or cell sample from the same individual, or alternatively, a non-tumor tissue or cell sample from a different individual. A detected pattern for a polypeptide is referred to as decreased in the mammalian tumor, tissue, or cell sample, if there is less polypeptide detected as compared to the a non-tumor tissue or cell sample. A detected pattern for a polypeptide is referred to as “increased” in the mammalian tumor, tissue, or cell sample, if there is more polypeptide detected as compared to the a non-tumor tissue or cell sample. A detected pattern for a polypeptide is referred to as “normal” in the mammalian tumor, tissue, or cell sample, if there is the same, or approximately the same, polypeptide detected as compared to a non-tumor tissue or cell sample.

The methods of this invention for identifying mammalian tumors that respond, or that do not respond, to an mTOR pathway inhibitor or a dual mTOR pathway inhibitor and EGF pathway inhibitor therapy comprise the step of assaying a sample obtained from the mammalian tumor to detect a pattern of expression, phosphorylation or both of one or a plurality of polypeptides consisting of: (a) HIF-1α polypeptide; (b) mTOR polypeptide; (c) phosphorylated MEK polypeptide; (d) phosphorylated ERK polypeptide. The combination of polypeptides and pattern of expression, phosphorylation, or both expression and phosphorylation identifies mammalian tumors that respond, or that do not respond, to an mTOR pathway inhibitor or a dual mTOR pathway inhibitor and EGF pathway inhibitor therapy -directed therapy. The methods can include the detection of a pattern of expression, phosphorylation or both of one, two, three, or all four of these polypeptides. Further, the methods can, but need not, include other steps, including steps such as the detection of a pattern of expression, phosphorylation or both of different polypeptides. Further, the methods can, but need not, include other steps, including steps such as the detection of a pattern of expression, phosphorylation or both of different polypeptides.

For example, the pattern that identifies a mammalian tumor as responding or that can be used to select a subject with cancer for treatment with a molecule targeted to a dual mTOR pathway inhibitor and EGF pathway inhibitor therapy is increased expression of the phosphorylated form of the ERIE polypeptide as compared to a non-tumor tissue or cell sample. Alternatively, the detected pattern is increased expression of the phosphorylated form of the MEK polypeptide as compared to a non-tumor tissue or cell sample. These identified patterns are understood to be non-limiting.

For example, the pattern that identifies a mammalian tumor as not responding or that can be used to select a subject with cancer to not receive treatment with a molecule targeted to an mTOR pathway inhibitor is increased expression of the phosphorylated form of the MEK polypeptide. Alternatively, the detected pattern is increased expression of the phosphorylated form of the MEK polypeptide as compared to a non-tumor tissue or cell sample. These identified patterns are understood to be non-limiting.

In practicing the methods of this invention, staining procedures can be carried out by a person, such as a histotechnician in an anatomic pathology laboratory. Alternatively, the staining procedures can be carried out using automated systems, such as Ventana Medical Systems' Benchmark® series of automated stainers. In either case, staining procedures for use according to the methods of this invention are performed according to standard techniques and protocols well-established in the art.

By “cell or tissue sample” is meant biological samples comprising cells, most preferably tumor cells, that are isolated from body samples, such as, but not limited to, smears, sputum, biopsies, secretions, cerebrospinal fluid, bile, blood, lymph fluid, urine and feces, or tissue which has been removed from organs, such as breast, lung, intestine, skin, cervix, prostate, and stomach. For example, a tissue samples can comprise a region of functionally related cells or adjacent cells.

The amount of target protein may be quantified by measuring the average optical density of the stained antigens. Concomitantly, the proportion or percentage of total tissue area stained can be readily calculated, for example as the area stained above a control level (such as an antibody threshold level) in the second image. Following visualization of nuclei containing biomarkers, the percentage or amount of such cells in tissue derived from patients after treatment are compared to the percentage or amount of such cells in untreated tissue. For purposes of the invention, “determining” a pattern of expression, phosphorylation, or both expression and phosphorylation of polypeptides is understood broadly to mean merely obtaining the expression level information on such polypeptide(s), either through direct examination or indirectly from, for example, a contract diagnostic service.

Alternatively, the amount of target protein can be determined using fluorescent methods. For example, Quantum dots (Qdots) are becoming increasingly useful in a growing list of applications including immunohistochemistry, flow cytometry, and plate-based assays, and may therefore be used in conjunction with this invention. Qdot nanocrystals have unique optical properties including an extremely bright signal for sensitivity and quantitation; high photostability for imaging and analysis. A single excitation source is needed, and a growing range of conjugates makes them useful in a wide range of cell-based applications. Qdot Bioconjugates are characterized by quantum yields comparable to the brightest traditional dyes available. Additionally, these quantum dot-based fluorophores absorb 10-1000 times more light than traditional dyes. The emission from the underlying Qdot quantum dots is narrow and symmetric which means overlap with other colors is minimized, resulting in minimal bleed through into adjacent detection channels and attenuated crosstalk, in spite of the fact that many more colors can be used simultaneously. Standard fluorescence microscopes are an inexpensive tool for the detection of Qdot Bioconjugates. Since Qdot conjugates are virtually photo-stable, time can be taken with the microscope to find regions of interest and adequately focus on the samples. Qdot conjugates are useful any time bright photo-stable emission is required and are particularly useful in multicolor applications where only one excitation source/filter is available and minimal crosstalk among the colors is required. For example, Quantum dots have been used as conjugates of Streptavidin and IgG to label cell surface markers and nuclear antigens and to stain microtubules and actin (Wu, X. et al. (2003). Nature Biotech. 21, 41-46).

As an example, Fluorescence can be measured with the multispectral imaging system Nuance™ (Cambridge Research & Instrumentation, Woburn, Mass.). As another example, fluorescence can be measured with the spectral imaging system SpectrView™ (Applied Spectral Imaging, Vista, Calif.). Multispectral imaging is a technique in which spectroscopic information at each pixel of an image is gathered and the resulting data analyzed with spectral image-processing software. For example, the Nuance system can take a series of images at different wavelengths that are electronically and continuously selectable and then utilized with an analysis program designed for handling such data. The Nuance system is able to obtain quantitative information from multiple dyes simultaneously, even when the spectra of the dyes are highly overlapping or when they are co-localized, or occurring at the same point in the sample, provided that the spectral curves are different. Many biological materials autofluoresce, or emit lower-energy light when excited by higher-energy light. This signal can result in lower contrast images and data. High-sensitivity cameras without multispectral imaging capability only increase the autofluorescence signal along with the fluorescence signal. Multispectral imaging can unmix, or separate out, autofluorescence from tissue and, thereby, increase the achievable signal-to-noise ratio.

In reference to antibody detection methods, “detection reagents” are meant reagents that can be used to detect antibodies, including both primary or secondary antibodies. For example, detection reagents can be fluorescent detection reagents, qdots, chromogenic detection reagents, or polymer based detection systems. However, the methods and kits of the invention are not limited by these detection reagents, nor are they limited to a primary and secondary antibody scheme (for example, tertiary, etc. antibodies are contemplated by the methods of the invention).

The present invention may also use mijcleic acid probes as a means of indirectly detecting the expressed protein biomarkers. For example, probes for the pERK, pMEK, HIF- 1α, and mTOR biomarkers can be constructed using standard probe design methodology, well-know to one of ordinary skill in the probe design art. As an example, U.S. Patent application Ser. No. US20050137389A1, “Methods and compositions for chromosome-specific staining,” incorporated by reference herein, describes methods of designing repeat-free probe compositions comprising heterogeneous mixtures of sequences designed to label an entire chromosome.

Gene-specific probes may be designed according to any of the following published procedures. To this end it is important to produce pure, or homogeneous, probes to minimize hybridizations at locations other than at the site of interest (Henderson, 1982, International Review of Cytology, 76:1-46). Manuelidis et al., (1984) Chromosoma, 91: 28-38, discloses the construction of a single kind of DNA probe for detecting multiple loci on chromosomes corresponding to members of a family of repeated DNA sequences.

Wallace et al., (1981), Nucleic Acids Research, 9:879-94, discloses the construction of synthetic oligonucleotide probes having mixed base sequences for detecting a single locus corresponding to a structural gene. The mixture of base sequences was determined by considering all possible nucleotide sequences that could code for a selected sequence of amino acids in the protein to which the structural gene corresponded.

Olsen et al., (1980) Biochemistry, 19:2419-28, discloses a method for isolating labeled unique sequence human X chromosomal DNA by successive hybridizations: first, total genomic human DNA against itself so that a unique sequence DNA fraction can be isolated; second, the isolated unique sequence human DNA fraction against mouse DNA so that homologous mouse/human sequences are removed; and finally, the unique sequence human DNA not homologous to mouse against the total genomic DNA of a human/mouse hybrid whose only human chromosome is chromosome X, so that a fraction of unique sequence X chromosomal DNA is isolated.

Cancer tissue sections taken from patients are analyzed, according to the methods of this invention by immunohistochemistry for expression, phosphorylation, or expression and phosphorylation of members of the mTOR pathway or the EGF pathway or any positive treatment response predictive combination thereof. In the methods of the invention, a change in “expression” can mean a change in number of cells in which the biomarker is detected, or alternatively, the number of positive cells may be the same, but the intensity (or level) may be altered. The term expression can be used as a surrogate term indicating changes in levels of molecular activation level.

These measurements can be accomplished, for example, by using tissue microarrays. Tissue microarrays are advantageously used in the methods of the invention, being well-validated method to rapidly screen multiple tissue samples under uniform staining and scoring conditions. (Hoos et al., 2001, Am JPathol. 158: 1245-51). Scoring of the stained arrays can be accomplished manually using the standard 0 to 3+ scale, or by an automated system that accurately quantifies the staining observed. The results of this analysis identify biomarkers that best predict patient outcome following treatment. Patient “probability of response” ranging from 0 to 100 percent can be predicted based upon the expression, phosphorylation or both of a small set of ligands, receptors, signaling proteins or predictive combinations thereof. Additional samples from cancer patients can be analyzed, either as an alternative to or in addition to tissue microarray results. For example, analysis of samples from breast cancer patients can confirm the conclusions from the tissue arrays, if the patient's responses correlate with a specific pattern of receptor expression and/or downstream signaling.

The invention provides, in part, kits for carrying out the methods of the invention. For example, the method provides kits for characterizing a mammalian tumor's responsiveness to an inhibitor of the mTOR pathway or a dual mTOR pathway inhibitor and an EGR pathway inhibitor comprising at least two reagents, preferably antibodies, that can detect the expression, phosphorylation, or both of polypeptides in the EGF pathway, the mTOR pathway, or both. For example, the kit can contain at least two, three, or four reagents that bind to a phosphorylated form of ERK, that bind to the phosphorylated form of MEK, that bind to HIF-1α , or that bind to mTOR. Further, the kit can include additional components other then the above-identified reagents, including but not limited to additional antibodies. Such kits may be used, for example, by a clinician or physician as an aid to selecting an appropriate therapy for a particular patient.

Particularly useful embodiments of the present invention and the advantages thereof can be understood by referring to Examples 1-6. These Examples are illustrative of specific embodiments of the invention, and various uses thereof. They are set forth for explanatory purposes only, and are not to be taken as limiting the invention.

### EXAMPLE 1

### Immunohistochemical Staining of Downstream Molecules in EGF/mTOR Pathways Under Hypoxic Conditions

The effect of hypoxia on the proteins downstream of the receptors in the EGF and rnTOR. pathways were assessed by evaluating the expression levels of markers of these pathways including, mTOR, HIF-1α, as well as the phospho-forms of mTOR, TSC2, S6, AKT, MEK, ERK (p44/p42), and 4EBP1. These markers were evaluated by immunohistochemistry (“IHC”) and image analysis in the presence of Desferrioxarnine (“DFO”)-induced hypoxia, as well as in its absence, or normoxia. DFO is an iron-chelating agent known to induce hypoxia, and was used in these experiments as a model for hypoxia. All IHC analyses were carried out on either the BenchMark XT® or Discovery XT® (Ventana Medical Systems, Inc., Tucson Ariz. (“VMSI”)) staining platforms. Primary antibodies were obtained from commercial sources (See Table 1). Controls were vehicle treated.

Jurkat (American Type Culture Collection (“ATCC”), Manasass, Va., Accession No. TIB-152) and HT1080 (ATCC CCL-121) cells lines were grown overnight either in the presence or absence of 50 μM DFO as models of hypoxia and normoxia. Cells were harvested and fixed in 10% neutral buffered forrnalin (“NBF”) and then paraffin-embedded (“FFPE”). FFPE cells were centrifuged for 10 min at 1500 rpm. Supernatant was removed and 3 drops of reagent 1 of the Shandon Cytoblock® Cell Block Preparation System (“Shandon Cytoblock”) (Thermo Electron Corporation, Waltham, Mass.) was added. Cells were centrifuged for 2 min at 3000 rpm. Three (3) drops of Shandon Cytoblock reagent 2 were dripped down the side of tube to allow reagent 2 to flow under the cell pellet suspension. Samples were incubated for 10 min, and then 5 ml of 70% ethanol was added (pellet floated to top of ethanol). Finally, samples were spun for 2 min at 3000 rpm, then transferred to a biopsy cassette and processed for paraffin embedding.

Hematoxylin and Eosin (“H&E”) staining was reviewed to verify suitability of the sections for IHC. H&E staining comprised the following steps: deparaffinizing in xylene, 100% ethanol and 95% ethanol, then immersion in water. Slides were immersed in hematoxylin for 3 min, rinsed in water, immersed in bluing reagent for 1 min, rinsed in water, dipped in eosin and finally a coverslip was added.

Immunoassays involved the following steps: antigen unmasking, and detection subsequent to incubation with the relevant primary and secondary antibodies. As a negative control, either the BenchMark XT® or Discovery XT® Diluent (VMSI) was incubated with the relevant slides. Primary antibodies were detected using the DABMap™, OmniMap™ (Discovery XT®), or iView™ DAB (BenchMark XT®) detection kit according to the manufacturer's instructions. Briefly, iVIEW™ DAB Detection Kit detected specific mouse IgG, IgM and rabbit IgG antibodies bound to an antigen in paraffin-embedded or frozen tissue section. The specific antibody was located by a biotin-conjugated secondary antibody. This step was followed by the addition of a streptavidin-enzyme conjugate that bound the biotin present on the secondary antibody. The complex was then visualized utilizing a precipitating chromogenic enzyme product. At the end of each incubation step, the automated slide stainer washed the sections to remove unbound material and applied a liquid coverslip that minimized evaporation of aqueous reagents from the slide. Results were interpreted using a light microscope and aided in the differential diagnosis of pathophysiological processes, which may or may not have been associated with a particular antigen. A summary of the developed protocols is presented in Table 1.

TABLE 1

| | | Primary | | |
|---|---|---|---|---|
| Primary | Antibody | Incubation/ | Detection | |
| Antibody | Source | titer | System | Vendor |
| mTOR | rabbit | 1 hr @ | iViewDAB ™ | Abcam 2732 |
| | polyclonal | 1:40 | | |
| HIF-1α | mouse | 1 hr @ | OmniMAP ™ | VMSI RUO |
| | monoclonal | 1:20 | | #760-4285 |
| pmTOR | rabbit | 1 hr @ | iViewDAB ™ | CST 2971 |
| | polyclonal | 1:10 | | |
| pTSC2 | rabbit | 1 hr @ | iViewDAB ™ | CST 3614 |
| | polyclonal | 1:2.5 | | |
| pS6 | rabbit | 32 min @ | iViewDAB ™ | CST 2211 |
| | polyclonal | 1:120 | | |
| pAKT | rabbit | 1 hr @ | DABMap ™ | CST 3787 |
| | monoclonal | 1:2.5 | | |
| pMEK | rabbit | 1 hr @ | iViewDAB ™ | CST 9121 |
| | polyclonal | 1:40 | | |
| pERK | rabbit | 1 hr | DABMap ™ | VMSI RUO |
| (p44/p42) | polyclonal | predilute | | #760-4228 |
| p4EBP1 | rabbit | 1 hr @ | iViewDAB ™ | CST 9455 |
| | polyclonal | 1:5 | | |

As a specific example, the detection of phospho-S6 (“pS6”) was accomplished in the following manner. H&Es' were reviewed by a pathologist to verify tumor presence for tissues and cell viability for cell lines and tissues. Primary antibody 2211 was obtained from Cell Signaling Technology, Inc. (“CST”) (Danvers, Mass.).

For the pS6 IHC assay, cell conditioning was carried out with CC1 conditioning buffer for 60 minutes at 100° C., where CC1 is a high pH cell conditioning solution: Tris/Borate/EDTA buffer, pH8 (VMSI). Slides were incubated with a 1/120 dilution of the stock concentration (See Table 1) of the primary antibody for 32 minutes at room temperature. Stock antibody concentration refers to the concentration at which the antibody is sold commercially; this information is not made available by some manufacturers. As a negative control, VMSI antibody diluent, used in accordance with manufacture's instructions, was incubated with the relevant slides under the same conditions. pS6 antibody was detected using the VMSI iView DAB detection kit with the exception of the universal secondary antibody, which was replaced by the Vector biotinylated anti-rabbit IgG, according to the manufacturer's instructions (Vector Laboratories, Burlingame, Calif.) and applied for 32 minutes at 37° C. Enzymatic detection/localization of pS6 was accomplished with a streptavidin horseradish peroxidase conjugate (VMSI), followed by reaction with hydrogen peroxide in the presence of diaminobenzidine (“DAB”) and copper sulfate, according to the manufacture's instructions and the kit used (see Table 1). The conjugate and all chromogenic reagents, with the exception of the Vector biotinylated secondary rabbit antibody, are also components of the iView detection kit and were applied at times recommended by the manufacturer.

Manual scoring was conducted by Board-certified pathologists. Staining intensities, percentage of reactive cells, and cellular localization were recorded. For qualitative stain intensity, 0 is the most negative and 3+ is the most positive. The principles of scoring used by the pathologists are outlined in the VMSI Pathway™ HER2/neu Scoring Guide. Slides were reviewed and scored by the pathologist prior to quantitation by optical imaging.

For optical imaging, a digital application (VMSI) with image quantification based on the intensity (expressed as average optical density, or avg. OD) of the stain converted to a numerical score was utilized. A high-resolution image was captured for each sample and the OD value was determined based on specific classifiers for the shape and color range for positively stained cells. At least three different areas per specimen were captured using either a 20× or 40× objective lens. In some cases, a “combined score” or multiplicative index was derived that incorporates both the percentage of positive cells and the staining intensity according to the following formula:
Combined score=(% positive)×(optical density score).

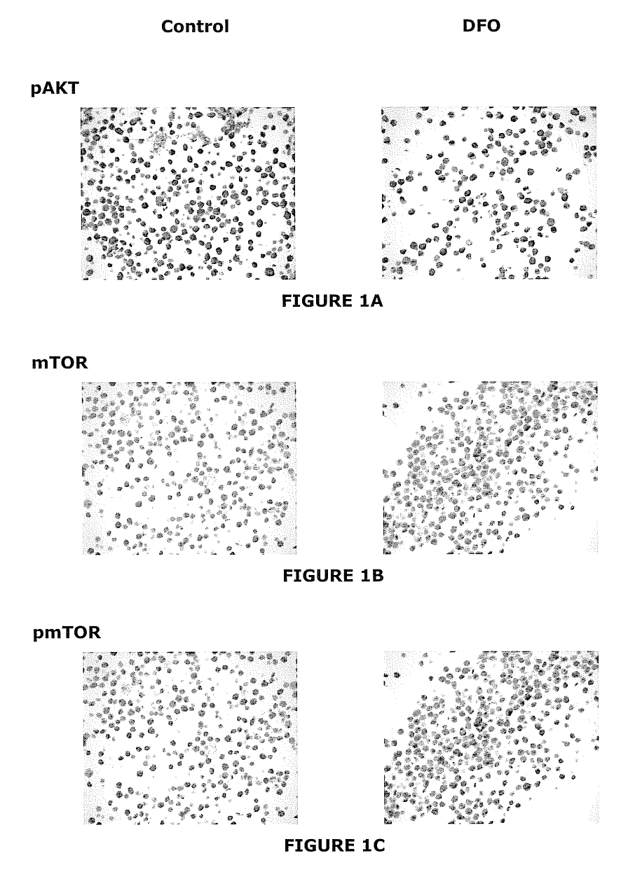

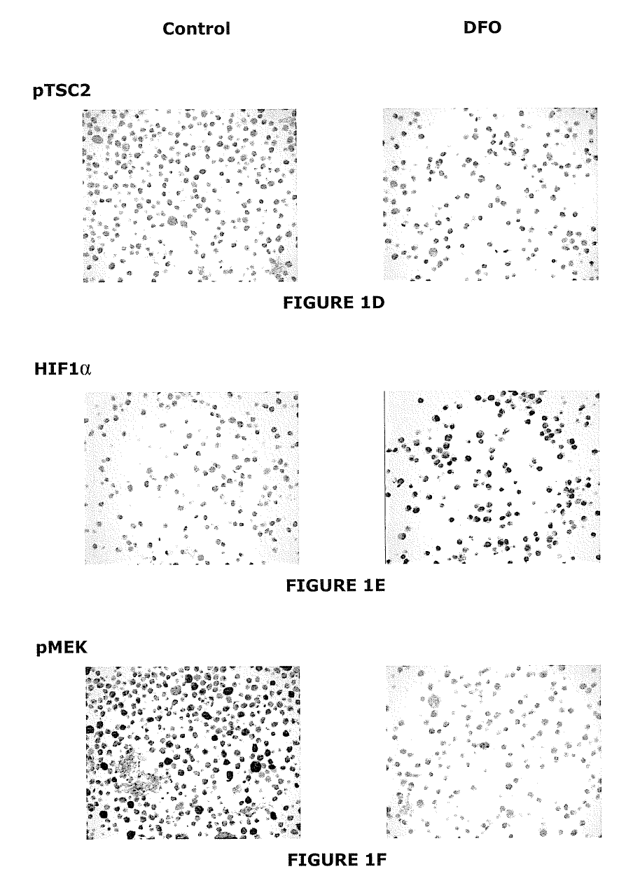

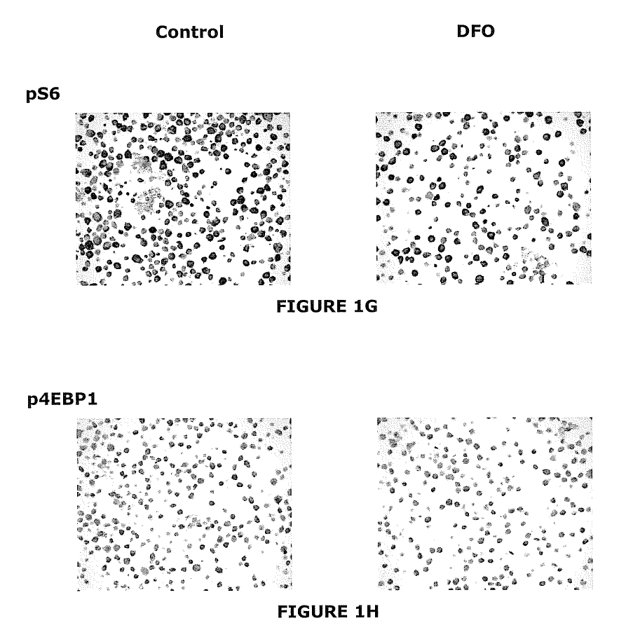

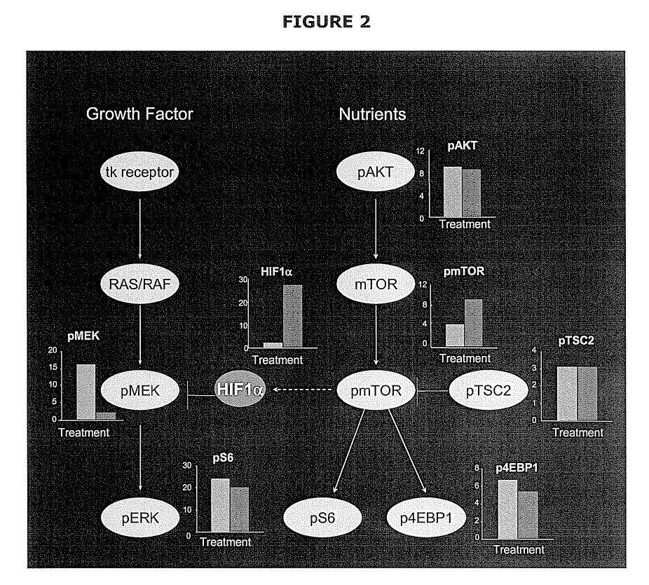

Representative images from Jurkat cells are illustrated in FIG. 1A-H and results are illustrated in FIG. 2. Induced-hypoxia resulted in a decrease in expression in p4EBP1, pMEK and pS6 and an increase in expression in HIF-1α and pmTOR, where expression is considered a measure of either number of cells in which the phospho-marker is detected or staining intensity. In fact, pMEK expression was not detectable in the presence of HIF-1α. There was no change in level of expression or activation of pAKT and pTSC2. No change was detected in the level of mTOR, which was consistently high, or pERK, which was negative in Jurkat cells and positive in HT1080 cells. These results demonstrated the utility of IHC methods for conducting rapid and reproducible staining procedures in a high-throughput and quantifiable format.

### EXAMPLE 2

### Expression and Inhibition of HIF1α in Response to Hypoxia

Jurkat cells and HT1080 cells were prepared for IHC as stated in Example 1 with DFO or vehicle treatment. Additionally, the HT1080 cells were treated in a dose escalation series with PX-478 (Pro1X Pharmaceuticals, Corp., Tucson, Ariz.), a HIF-1α inhibitor (small molecule). Controls were vehicle treated. The conditions are summarized in Table 2.

TABLE 2

| Specimen Type | Treatment Samples |
|---|---|
| Jurkat Cell Line | Vehicle |
| | DFO Treated |
| HT1080 Cell Line | Vehicle |
| | DFO Treated |
| | DF0 + 25 μm HIF1 Inhibitor Treated |
| | DF0 + 50 μm HIF1 Inhibitor Treated |
| | DF0 + 75 μm HIF1 Inhibitor Treated |

IHC was conducted and assessed according to the procedures detailed in Example 1. Western Blotting and FACS analysis were conducted using standard conditions.

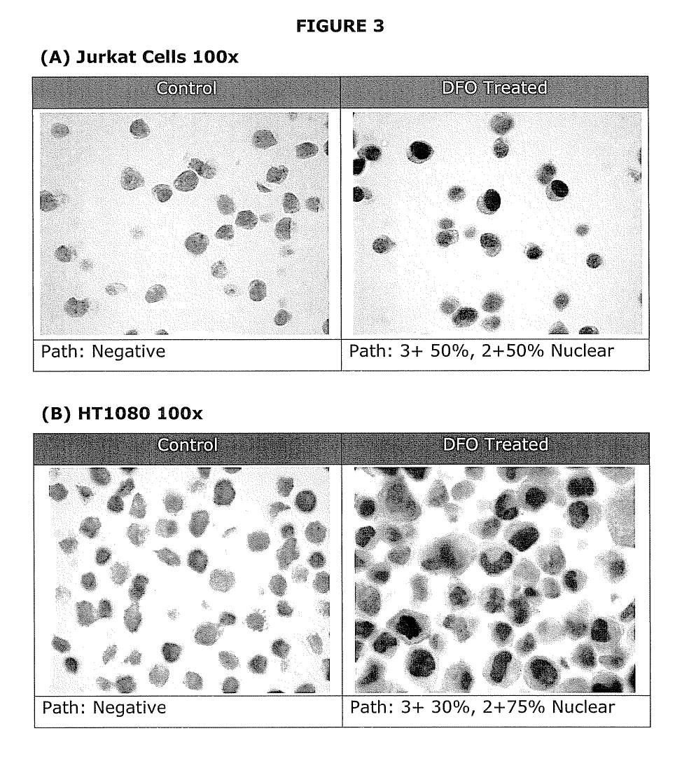

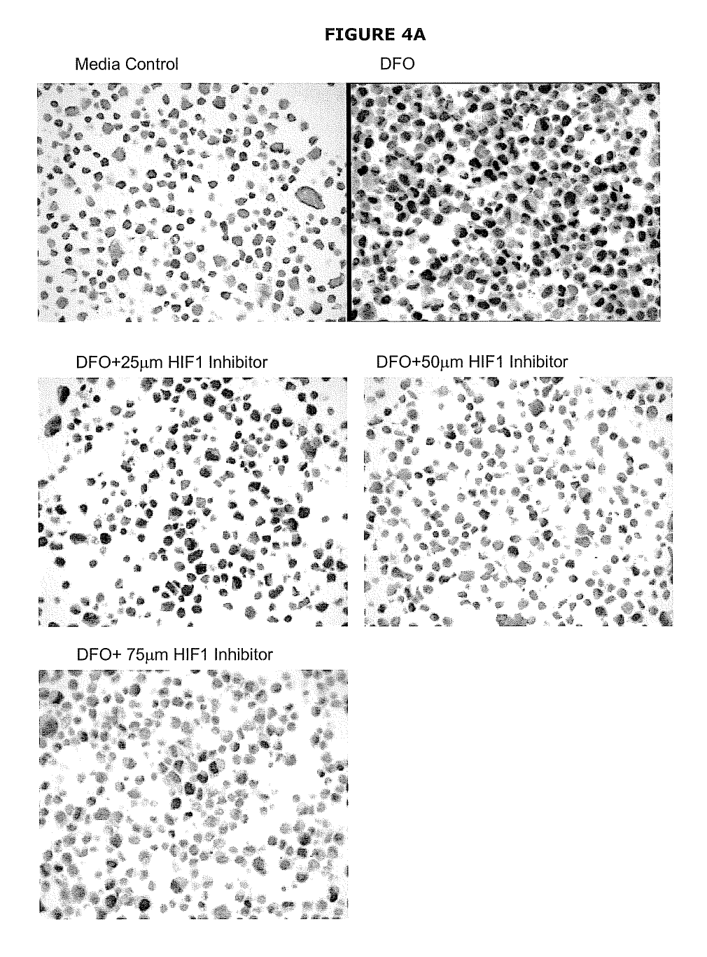

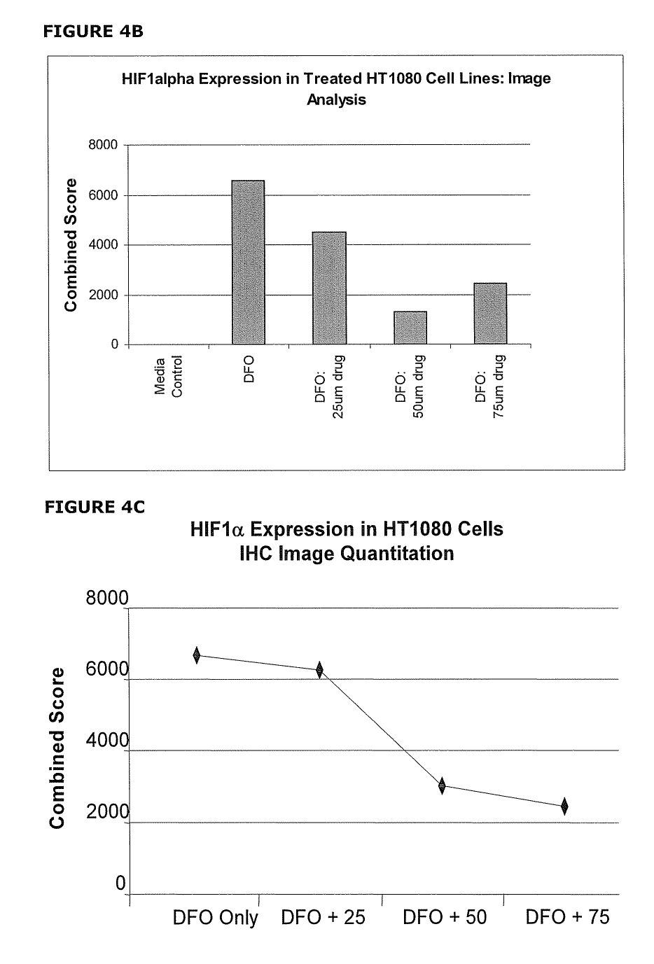

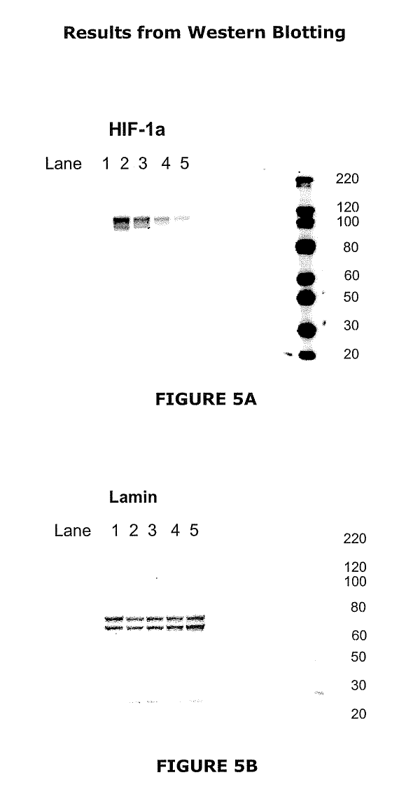

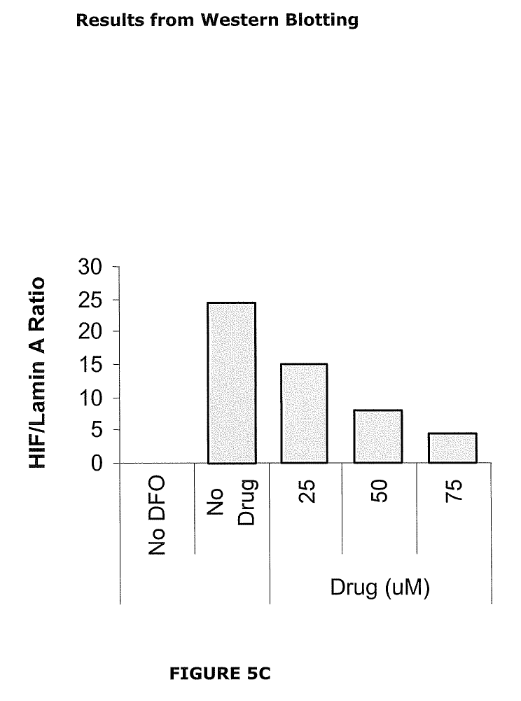

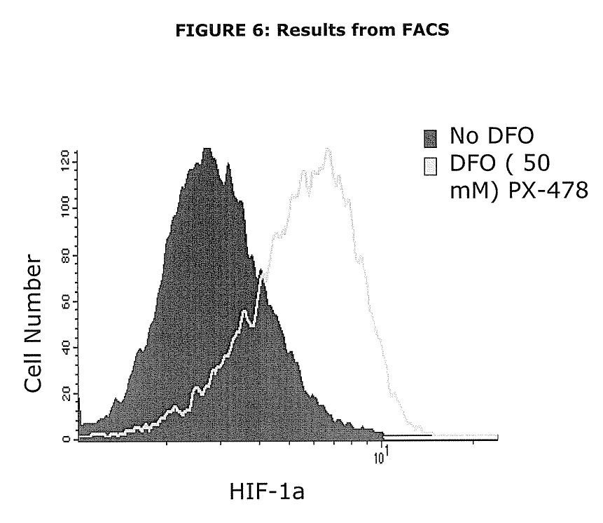

DFO treatment resulted in increased expression of HIF-1α in both Jurkat and HT1080 cell lines. Representative staining images are shown in FIGS. 3A (Jurkat) and FIG. 3B (HT1080). HIF-1α inhibitor treatment (see Table 2) resulted in a concentration-dependent decrease in HIF-1α expression in response to DFO-induced hypoxia. FIG. 4A shows representative images at each inhibitor concentration (25 μM, 50 μM, and 75 μM PX-478). FIGS. 4B and 4C show the results of image analysis of HIF-1α levels for each treatment group. Western blot analysis further demonstrated this decrease in levels of HIF-1α with increasing concentration of HIF-1α inhibitor in response to DFO induced hypoxia, as represented in FIG. 5A (Lanes (1) Vehicle-treated, (2) DFO, (3) DFO+25 μM, (4) DFO+50 μM, and (5) DFO+75 μM PX-478)). Laminin detection was used to determine equivalent loading (FIG. 5B). Quantification of HIF-1α expression levels was determined by densitometry in the presence of the inhibitor (FIG. 5C). The results from FACS further confirm an increase in HIF-1α after treatment with DFO (FIG. 6). These results confirm that HIF-1α levels are increased in response to hypoxia, and correspondingly that levels of hypoxia-induced HIF-1α are decreased in the presence of the inhibitor.

### EXAMPLE 3

### Modulation of EGF Downstream Markers in Response to HIF1α Inhibition under Hypoxic Conditions

To assess the interaction between the EGF and mTOR pathways, expression (where expression is considered a measure of either number of cells in which the phospho-marker is detected or staining intensity) of pMEK and pERK, downstream markers in the EGF pathway was measured in response to the HIF-1α inhibitor PX-478 under hypoxic conditions. HT1080 cells were prepared for IHC as described in Example 1 with DFO or vehicle treatment and in the presence of increasing concentration of HIF-1α inhibitor (see Table 2). IHC staining for pMEK, pERK, pAKT, and pS6 was performed as described in Example 1.

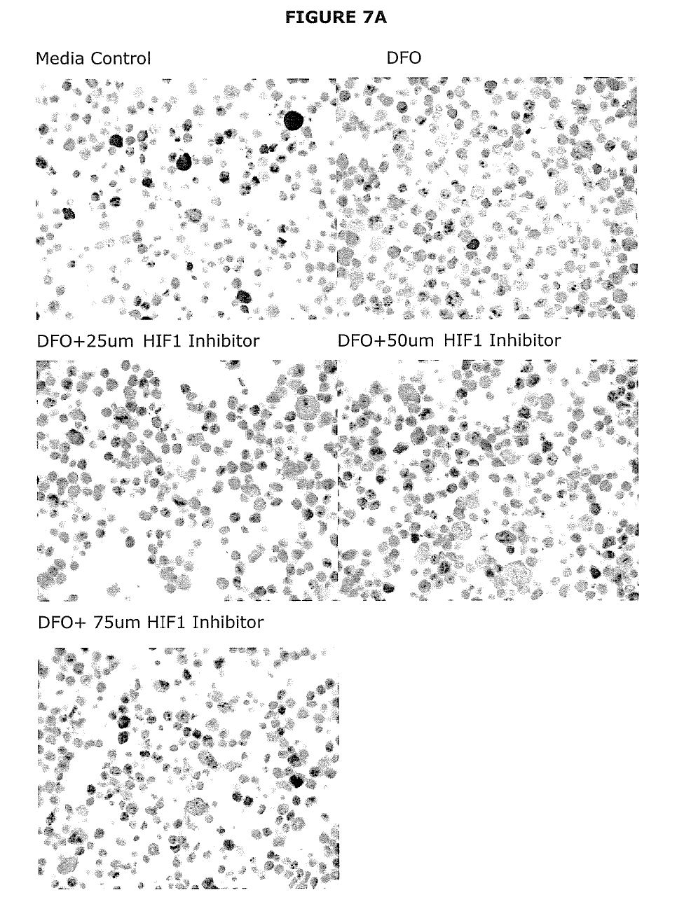

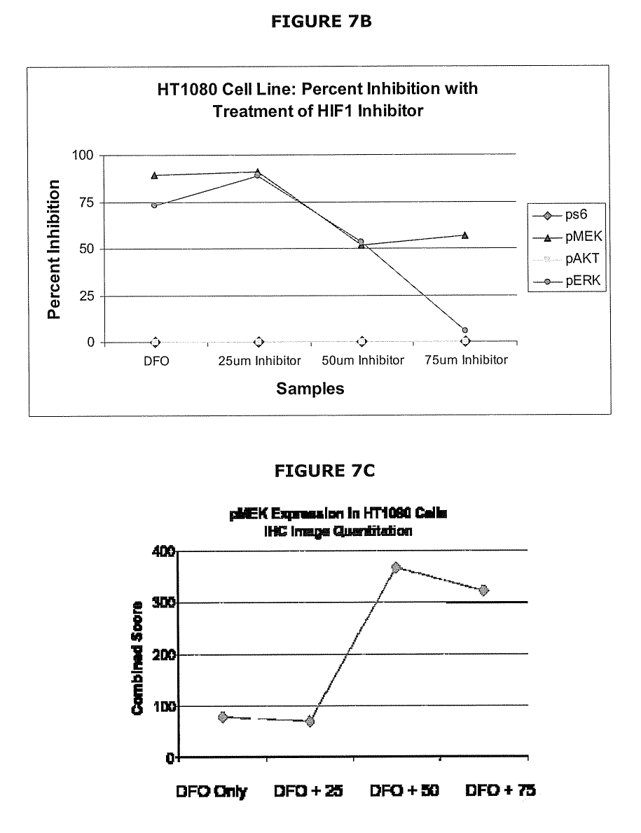

FIG. 7A shows representative images of increased pMEK staining with increasing concentration of HIF-1α inhibitor (25 μM, 50 μM, and 75 μM PX-478). The level of expression inhibition of pMEK and pERK in response to hypoxia was reduced with increasing HIF-1α inhibitor, while pS6 and pAKT expression was not altered (FIG. 7B). FIG. 7C represents the increase in pMEK combined score (detailed in Example 1) with increasing concentrations of HIF-1α inhibitor. These results showed that markers of the EGF pathway (pMEK and pERK) were increased in response to increasing concentrations of HIF-1α inhibitor.

### EXAMPLE 4

### Double Labeling of HIF-1α and Downstream Markers of the mTOR Pathway

In order to simultaneously monitor changes in the EGF and mTOR pathways in the same tissue, IHC double labeling was performed for HIF-1α and pMEK or HIF-1α and pERK. HeLa human tumor cell xenograft samples were produced according to the protocol previously described in co-owned and co-pending U.S. Ser. No. 11/416,362, filed May 1, 2006, incorporated herein by reference. The HeLA cell xenografts were then analyzed on VMSI Discovery® XT as described in Example 5. IHC was performed as previously described in Example I with primary antibodies titers and incubation times for HIF-1α, pMEK, and pERK as listed in Table 1.

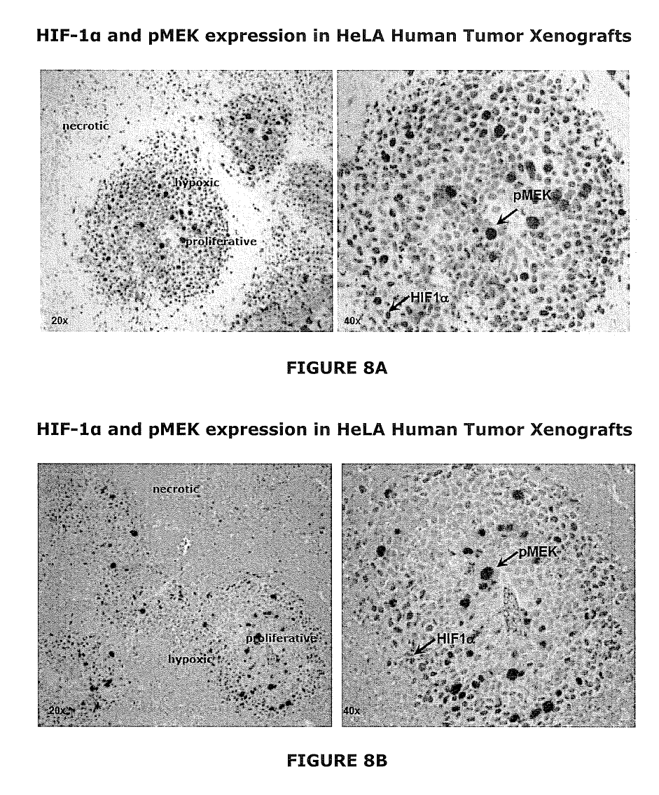

Two different types of detecting secondary antibodies and detection substrates were used to visualize the staining: Dual Brightfield IHC and QDOT Fluorescence. For Dual Brightfield IHC, the secondary antibodies were directly conjugated to horseradish peroxidase (“HRP”) (UltraMAP P.N. 760-500, VMSI) or alkaline phosphatase (“AP”). The detection substrates were DAB/Copper (ChromoMap DAB, VMSI) for HRP and Nuclear Fast Red (ChromoMap RED, VMSI) or NBT/BCIP (ChromoMap BLUE, VMSI) for AP and were used according to mamifacture's instructions. FIG. 8 represents the results from the Dual Brightfield IHC labeling for (A) HIF-1α (detected using Nuclear Fast Red) and pMEK (detected using DAB-brown) and (B) HIF-1 α (detected using DAB-brown) and pMEK (detected using NBT/BCIP-Blue). The respective biomarkers are labeled in FIGS. 8A-B.

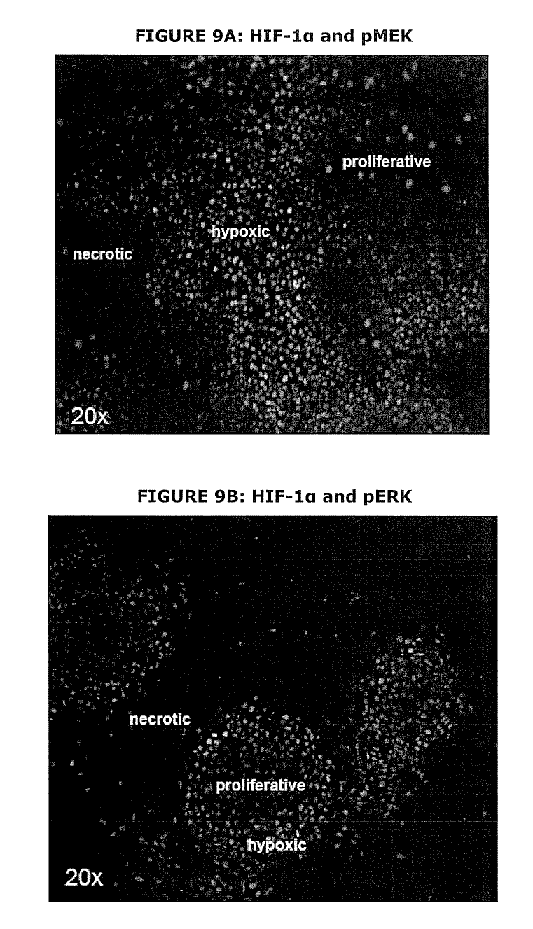

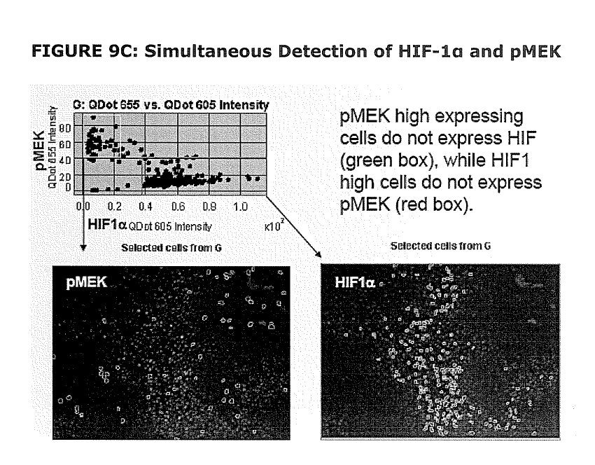

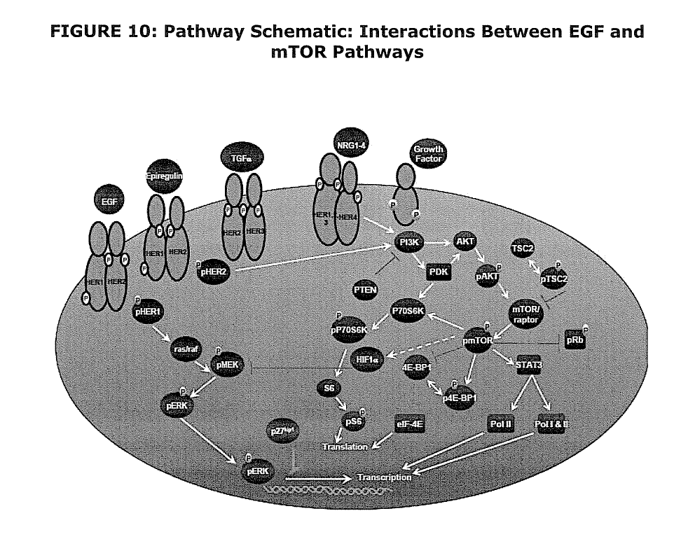

For QDOT Fluorescent IHC, secondary antibodies were the same as those described in Example 1 and detection substrates were streptavidin-conjugated QDOT (VMSI QD605 or QD655). Image analysis was performed by initially capturing image cubes on a spectral imaging camera (Cambridge Research Instruments, Woburn, Mass.). Excitation was conducted with a UV (mercury) light source. The image cubes were then analyzed on VMSI Research Imaging Application. Briefly, image cubes were retrieved in the application and data was extracted and reported based on the pixel intensities of QDots expected to emit at 605 nm (HIF-1α) and 655 nm (pMEK or pERK). Analysis using the application was then conducted to identify individual HIF-1α and/or pMEK and/or pERK expressing cells in the tumor. FIG. 9 represents QDOT Fluorescent double labeling for HIF-1α and (A) pMEK or (B) pERK. The HIF-1α expression cells are found mainly in the hypoxic-labeled field in FIG. 9, while pMEK and pERK are mainly found in the proliferative-labeled field. As shown in the Figure, cells expressing high levels of pMEK were found not to express HIF-1α, while cells expressing high levels of HIF-1α did not express pMEK (FIG. 9C). FIG. 10 represents a schematic diagram of cross-talk between EGF and mTOR pathways suggested by these results.

### EXAMPLE 5

### HIF1α Assay Protocol

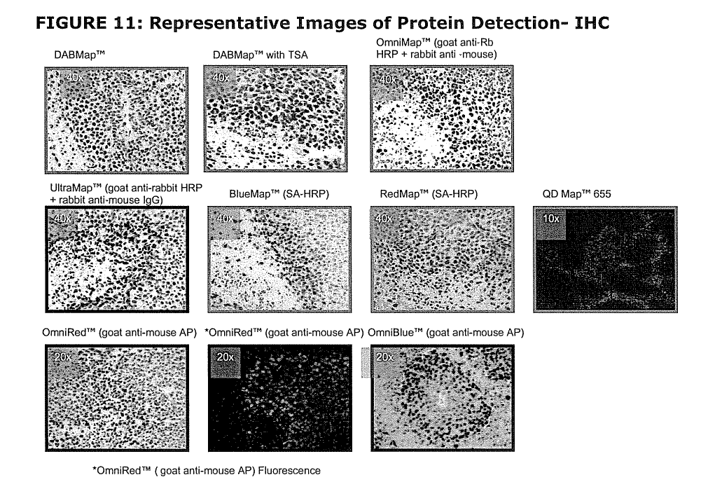

The anti-HIF-1α primary antibody assay protocol allows for rapid assessment of HIF-1α expression in tumor samples and changes in expression in response to cancer treatment. VMSI anti-HIF-1α (mouse monoclonal) Primary Antibody was tested on the Discovery®, Discovery® XT, BenchMark® and BenchMark® XT platforms and may be detected with the selected detection kits: DABMap™, BlueMap™, RedMap™, OmniMap™, AmpMap™, QDMap™655, iView™DAB, ultra™View. FIG. 11 shows representative images from the above detection kits. Assay specificity, range, and linearity were examined using a multi-organ tissue microarray containing cores of normal and neoplastic tissues. A pathologist's scoring of these samples was based on staining intensity, contrast-to-background, and sub-cellular localization to the nucleus. The antibody was tested in formalin-fixed paraffin embedded tissues and cell lines. The Discovery® Protocol Conditions are outlined below:
a. Open the NexES software.
b. To create a protocol, click on the “Protocols” button on the main screen. A window will appear on the screen with “Create/Edit Protocol” and “Manage Protocol”. Click on “Create/Edit Protocol” to open the “NexES Protocol Editor” window.
c. Select the appropriate procedure under the “Procedure” field.
d. Check the box next to “Tissue”
e. Check the box next to “Paraffin”
f. Check the box next to “Cell Conditioning”
g. Check the box next to “Conditioner #1”
h. Check the box next to “Mild CC 1”
i. Check the box next to “Standard CC 1”
j. Check the box next to “No Heat”
k. Check the box next to “Antibody”
l. Check the box next to “Antibody Auto Dispense”
m. Check the box next to “Standard Ab Incubation”
n. In the “Antibody” pull down menu select “anti-HIFα”
o. In the “Standard Ab Incubation Time” pull down menu select “1 hour”
p. Continue to check the appropriate boxes and make the appropriate selections for your secondary antibody and detection.
   The antibody was developed and optimized using the Discovery® OmniMAP™ line of detection with LinkOD™ and goat anti-rabbit HRP detection.

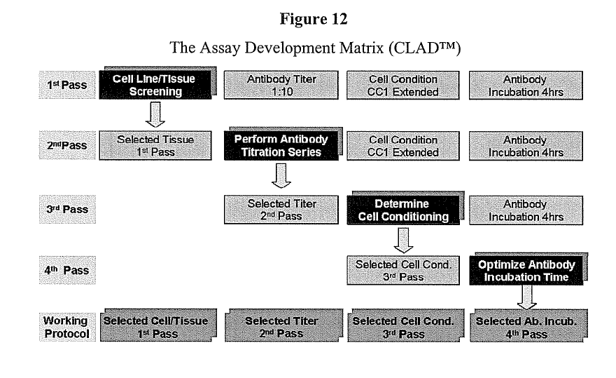

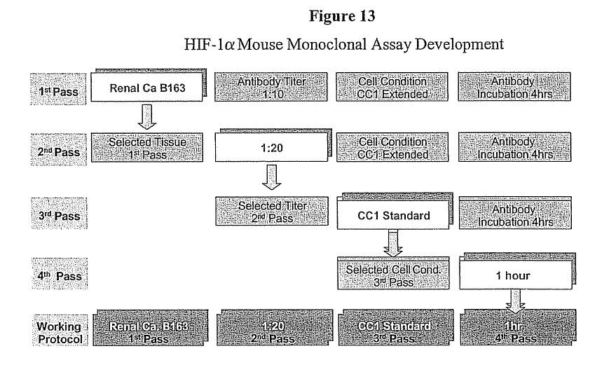

The rapid assay development process (FIG. 12) for the detection of the HIF- 1α in candidate samples is shown in FIG. 13. Table 3 summarizes the resulting Final Assay Protocol.

TABLE 3
Final Assay Protocol

| | |
|---|---|
| Tissue (selected 1^{st} Pass) | Renal Cell Carcinoma |
| Cell Condition (selected 3^{rd} Pass) | CC1 Standard |
| Titer (selected 2^{nd} Pass) | 1:20 |
| Antibody Incubation Time | 1 hour |
| (selected 4^{th} Pass) | |
| Instrument | Discovery ®XT |
| Secondary Antibody | Universal |
| Detection Kit | OmniMap ™ (goat anti-Rabbit |
| | HRP + LinkOD ™) |

### EXAMPLE 6

### Effects of mTOR Inhibition on HIF-1α and EGF Pathway Markers

HT1080, Jurkat, and a percentage of LNCaP (ATCC CRL-1740) (prostate carcinoma cells) HIF+/pMEK− cells are treated with rapamycin, an mTOR inhibitor. Inhibition of mTOR is expected to result in a decrease in HIF-1α and an induction of pMEK. Hypoxia is induced in susceptible human cell line models, including HT1080, Jurkat and LNCaP, by incubating 1×10⁶ cells/mL in 10 mL of cell culture media (RPMI supplemented with 10% fetal bovine serum and conventional amounts of antibiotics (penicillin and streptomycin) and L-Glutamate; GIBCO-BRL) with or without DFO (50μM) for 16 hours. After overnight culture, cells are washed twice in phosphate buffered saline (PBS), and incubated with culture media as defined above or media supplemented with various doses of rapamycin including (50 μM, 30μM, 10μM or media only) for an additional 18 hours. Post treatment, cells are washed 2× in PBS. Adherent cells (such as LnCAP) are trypsinized. All cultures are resuspended in media and centrifuged. Post-centrifugation, media is removed and cell are resuspended in 10% Phosphate Buffered Formalin. Cells are then fixed in 10% Phosphate Buffered Formalin for 4-6 hours, washed in PBS×2 Cells and fixed in 70% ethanol prior to embedding as described previously (Example 1). IHC are conducted for HIF-1α, mTOR, pmTOR, pS6, pMEK and pERK as described in Example 1 and Table 1. The induction of hypoxia should decrease pMEK and pERK, while the the treatment with rapamycin should decrease HIF levels, and correspondingly induce pMEK..

It should be understood that the foregoing disclosure emphasizes certain specific embodiments of the invention and that all modifications or alternatives equivalent thereto are within the spirit and scope of the invention as set forth in the appended claims.

What we claim is:

1. A method for identifying a mammalian tumor that can be treated with a dual mTOR pathway inhibitor and EGF pathway inhibitor therapy, comprising the step of assaying a sample obtained from the mammalian tumor to detect a pattern of expression, phosphorylation or both expression and phosphorylation of a panel of two or more polypeptides consisting of:
(a) at least one polypeptide of the EGF pathway, and
(b) at least one polypeptide of the mTOR pathway
wherein the expression, phosphorylation or both expression and phosphorylation of the panel of polypeptides identifies the mammalian tumor as treatable with a dual mTOR pathway inhibitor and EGF pathway inhibitor therapy.

2. The method of claim 1, wherein the at least one polypeptide of the EGF pathway comprises a phosphorylated ERK polypeptide; a phosphorylated MEK polypeptide, or both a phosphorylated ERK polypeptide and a phosphorylated MEK polypeptide.

3. The method of claim 2, wherein the mammalian tumor is identified as treatable with a duel mTOR pathway inhibitor and EGF pathway inhibitor therapy when the detected pattern of expression, phosphorylation or both expression and phosphorylation of the panel of polypeptides in the sample is greater than the level of expression, phosphorylation or both expression and phosphorylation of the panel of polypeptides in a non-tumor control.

4. The method of claim 1, wherein the at least one polypeptide of the mTOR pathway comprises HIF-1α polypeptide, mTOR polypeptide, or both HIF-1α and mTOR polypeptide.

5. The method of claim 4, wherein the mammalian tumor is identified as treatable with a duel niTOR pathway inhibitor and EGF pathway inhibitor therapy when the detected pattern of expression, phosphorylation or both expression and phosphorylation of the panel of polypeptides in the sample is greater than the level of expression, phosphorylation or both expression and phosphorylation of the panel of polypeptides in a non-tumor control.

6. The method of claim 1, wherein the at least one polypeptide of the EGF pathway comprises the phosphorylated ERK polypeptide; the phosphorylated MEK polypeptide, or both the phosphorylated ERK polypeptide and the phosphorylated MEK polypeptide; and wherein the at least one polypeptide of the mTOR pathway comprises HIF-1α polypeptide, mTOR polypeptide, or both HIF-1α and mTOR polypeptide.

7. The method of claim 6, wherein the mammalian tumor is identified as treatable with a duel mTOR pathway inhibitor and EGF pathway inhibitor therapy when the detected pattern of expression, phosphorylation or both expression and phosphorylation of the panel of polypeptides in the sample is greater than the level of expression, phosphorylation or both expression and phosphorylation of the panel of polypeptides in a non-tumor control.

8. The method of claim 1, wherein the at least one polypeptide of the EGF pathway comprises the phosphorylated ERK polypeptide; and wherein the at least one polypeptide of the mTOR pathway comprises HIF-1α polypeptide.

9. The method of claim 8, wherein the mammalian tumor is identified as treatable with a duel mTOR pathway inhibitor and EGF pathway inhibitor therapy when the detected pattern of expression, phosphorylation or both expression and phosphorylation of the panel of polypeptides in the sample is greater than the level of expression, phosphorylation or both expression and phosphorylation of the panel of polypeptides in a non-tumor control.

10. The method of claim 1, wherein the at least one polypeptide of the EGF pathway comprises the phosphorylated MEK polypeptide; and wherein the at least one polypeptide of the mTOR pathway comprises HIF-1α polypeptide.

11. The method of claim 10, wherein the mammalian tumor is identified as treatable with a duel mTOR pathway inhibitor and EGF pathway inhibitor therapy when the detected pattern of expression, phosphorylation or both expression and phosphorylation of the panel of polypeptides in the sample is greater than the level of expression, phosphorylation or both expression and phosphorylation of the panel of polypeptides in a non-tumor control.

12. A method for assessing a positive response to receiving a dual mTOR pathway inhibitor and EGF pathway inhibitor therapy in an individual, comprising
(a) obtaining a first tissue or cell sample from the individual before exposing the individual to the dual mTOR pathway inhibitor and EGF pathway inhibitor therapy;
(b) obtaining a second tissue or cell sample from the individual after exposing the individual to the dual mTOR pathway inhibitor and EGF pathway inhibitor therapy;
(c) detecting a pattern of expression, phosphorylation or both expression and phosphorylation of a panel of two or more polypeptides consisting of:
(i) at least one polypeptide of the EGF pathway, and
(ii) at least one polypeptide of the mTOR pathway in said first tissue or cell sample and said second tissue or cell sample;
(d) detecting a difference in the pattern of expression, phosphorylation or both expression and phosphorylation between the first tissue or cell sample and the second tissue or cell sample,
wherein decreased expression, phosphorylation or both expression and phosphorylation between the second tissue or cell sample and the first tissue or cell sample shows a positive response to receiving the dual mTOR pathway inhibitor and EGF pathway inhibitor therapy.

13. The method of claim 12, wherein the at least one polypeptide of the EGF pathway comprises a phosphorylated ERK polypeptide; a phosphorylated MEK polypeptide, or both a phosphorylated ERK polypeptide and a phosphorylated MEK polypeptide.

14. The method of claim 12, wherein the at least one polypeptide of the mTOR pathway comprises HIF-1α polypeptide, mTOR polypeptide, or both HIF-1α and mTOR polypeptide.

15. The method of claim 12, wherein the at least one polypeptide of the EGF pathway comprises a phosphorylated ERK polypeptide; a phosphorylated MEK polypeptide, or both a phosphorylated ERK polypeptide and a phosphorylated MEK polypeptide; and wherein the at least one polypeptide of the mTOR pathway comprises HIF-1α polypeptide, mTOR polypeptide, or both HIF-1α and mTOR polypeptide.

16. The method of claim 12, wherein the at least one polypeptide of the EGF pathway comprises a phosphorylated ERK polypeptide; and wherein the at least one polypeptide of the mTOR pathway comprises HIF- 1α polypeptide.

17. The method of claim 12, wherein the at least one polypeptide of the EGF pathway comprises a phosphorylated MEK polypeptide; and wherein the at least one polypeptide of the mTOR pathway comprises HIF-1α polypeptide.

18. A kit for identifying a mammalian tumor that can be treated with or assessing a positive response in an individual to receiving a dual mTOR pathway inhibitor and EGF pathway inhibitor therapy comprising at least two reagents for detecting the expression, phosphorylation or both expression and phosphorylation of polypeptides in the EGF pathway, the mTOR pathway, or both the EGF pathway and the mTOR pathway.

19. The kit of claim 18, wherein the at least two reagents detect the expression, phosphorylation or both expression and phosphorylation of a panel of polypeptides consisting of:
(a) at least one polypeptide of the EGF pathway, and
(b) at least one polypeptide of the mTOR pathway.

20. The kit of claim 18, wherein the at least one polypeptide of the EGF pathway is a phosphorylated form of ERK..

21. The kit of claim 18, wherein the at least one polypeptide of the EGF pathway is a phosphorylated form of MEK.

22. The kit of claim 18, wherein the at least one polypeptide of the mTOR pathway is HIF-1α.

23. The kit of claim 18, wherein the at least one polypeptide of the mTOR pathway is mTOR.

24. The kit of claim 18, wherein the at least one polypeptide of the EGF pathway is a phosphorylated form of ERK and wherein the at least one polypeptide of the mTOR pathway is HIF-1α.

25. The kit of claim 18, wherein the at least one polypeptide of the EGF pathway is a phosphorylated form of MEK and wherein the at least one polypeptide of the mTOR pathway is HIF-1α.

26. The kit of claim 18, wherein the at least two reagents are antibodies.

27. The kit of claim 18, wherein the at least two reagents comprise:
(a) at least one antibody that binds to an epitope of a polypeptide of the EGF pathway, and
(b) at least one antibody that binds to an epitope of a polypeptide of the mTOR pathway.

28. The kit of claim 27, further comprising a detection reagent.

29. The kit of claim 27, wherein the at least one antibody that binds to an epitope of a polypeptide of the EGF pathway binds to an epitope of ERK.

30. The kit of claim 27, wherein the at least one antibody that binds to an epitope of a polypeptide of the EGF pathway binds to an epitope of the phosphorylated form of ERK.

31. The kit of claim 27, wherein the at least one antibody that binds to an epitope of a polypeptide of the EGF pathway binds to an epitope of MEK.

32. The kit of claim 27, wherein the at least one antibody that binds to an epitope of a polypeptide of the EGF pathway binds to an epitope of the phosphorylated form of MEK.

33. The kit of claim 27, wherein the at least one antibody that binds to an epitope of a polypeptide of the mTOR pathway binds to an epitope of HIF-1α.

34. The kit of claim 27, wherein the at least one antibody that binds to an epitope of a polypeptide of the mTOR pathway binds to an epitope of mTOR.

35. The kit of claim 27, wherein the at least one antibody that binds to an epitope of a polypeptide of the EGF pathway binds to an epitope of ERK and wherein the at least one antibody that binds to an epitope of a polypeptide of the mTOR pathway binds to an epitope of HIF-1α.

36. The kit of claim 35, further comprising a detection reagent.

37. The kit of claim 27, wherein the at least one antibody that binds to an epitope of a polypeptide of the EGF pathway binds to an epitope of the phosphorylated form of ERK and wherein the at least one antibody that binds to an epitope of a polypeptide of the mTOR pathway binds to an epitope of HIF- 1α.

38. The kit of claim 37, further comprising a detection reagent.

39. The kit of claim 27, wherein the at least one antibody that binds to an epitope of a polypeptide of the EGF pathway binds to an epitope of MEK and wherein the at least one antibody that binds to an epitope of a polypeptide of the mTOR pathway binds to an epitope of HIF-1α.

40. The kit of claim 39, further comprising a detection reagent.

41. The kit of claim 27, wherein the at least one antibody that binds to an epitope of a polypeptide of the EGF pathway binds to an epitope of the phosphorylated form of MEK and wherein the at least one antibody that binds to an epitope of a polypeptide of the mTOR pathway binds to an epitope of HIF- 1α

42. The kit of claim 41, further comprising a detection reagent.

43. A therapeutic treatment comprising an inhibitor of the EGF pathway and an inhibitor of HIF- 1α.

44. The therapeutic treatment of claim 43, wherein the inhibitor of the EGF pathway is an inhibitor of MEK phosphorylation.

45. The therapeutic treatment of claim 43, wherein the inhibitor of the EGF pathway is an inhibitor of ERK phosphorylation.

46. The therapeutic treatment of claim 43, wherein the inhibitor of HIF-1α is PX-478.

47. A method for identifying a mammalian tumor that can be treated with a dual mTOR pathway inhibitor and EGF pathway inhibitor therapy, comprising the step of assaying a mammalian tumor sample obtained from an individual that has received an mTOR pathway inhibitor to detect a pattern of expression, phosphorylation or both expression and phosphorylation of at least one polypeptide of the EGF pathway; wherein the expression, phosphorylation or both expression and phosphorylation of the at least one polypeptide of the EGF pathway identifies the mammalian tumor as treatable with a dual mTOR pathway inhibitor and EGF pathway inhibitor therapy.

48. The method of claim 47, wherein the at least one polypeptide of the EGF pathway comprises a phosphorylated ERK polypeptide; a phosphorylated MEK polypeptide, or both a phosphorylated ERK polypeptide and a phosphorylated MEK polypeptide.

49. The method of claim 48, wherein the detected pattern of expression, phosphorylation or both expression and phosphorylation of the at least one polypeptide of the EGF pathway is compared to a pattern of expression, phosphorylation or both expression and phosphorylation of the at least one polypeptide of the EGF pathway in a control; wherein an increased levels of the at least one polypeptide of the EGF pathway in the sample as compared to the levels of the panel of polypeptides in the control identifies the mammalian tumor as treatable with a dual mTOR pathway inhibitor and EGF pathway inhibitor therapy.

50. The method of claim 47, wherein the at least one polypeptide of the EGF pathway comprises the phosphorylated ERK polypeptide.

51. The method of claim 50, wherein the detected pattern of expression, phosphorylation or both expression and phosphorylation of the phosphorylated ERK polypeptide is compared to a pattern of expression, phosphorylation or both expression and phosphorylation of the phosphorylated ERK polypeptide in a control; wherein an increased levels of the phosphorylated ERK polypeptide in the sample as compared to the levels the phosphorylated ERK polypeptide in the control identifies the mammalian tumor as treatable with a dual mTOR pathway inhibitor and EGF pathway inhibitor therapy.

52. The method of claim 47, wherein the at least one polypeptide of the EGF pathway comprises the phosphorylated MEK polypeptide.

53. The method of claim 52, wherein the detected pattern of expression, phosphorylation or both expression and phosphorylation of the phosphorylated MEK polypeptide is compared to a pattern of expression, phosphorylation or both expression and phosphorylation of the phosphorylated MEK polypeptide in a control; wherein an increased levels of the phosphorylated MEK polypeptide in the sample as compared to the levels the phosphorylated MEK polypeptide in the control identifies the mammalian tumor as treatable with a dual mTOR pathway inhibitor and EGF pathway inhibitor therapy.

54. A method for identifying a mammalian tumor that can be treated with a dual mTOR pathway inhibitor and EGF pathway inhibitor therapy, comprising the steps of (1) treating a mammalian tumor sample obtained from an individual with an mTOR pathway inhibitor; and (2) detecting a pattern of expression, phosphorylation or both expression and phosphorylation of at least one polypeptide of the EGF pathway; wherein the expression, phosphorylation or both expression and phosphorylation of the at least one polypeptide of the EGF pathway identifies the mammalian tumor as treatable with a dual mTOR pathway inhibitor and EGF pathway inhibitor therapy.

55. The method of claim 54, wherein the at least one polypeptide of the EGF pathway comprises a phosphorylated ERK polypeptide; a phosphorylated MEK polypeptide, or both a phosphorylated ERK polypeptide and a phosphorylated MEK polypeptide.

56. The method of claim 55, wherein the detected pattern of expression, phosphorylation or both expression and phosphorylation of the at least one polypeptide of the EGF pathway is compared to a pattern of expression, phosphorylation or both expression and phosphorylation of the at least one polypeptide of the EGF pathway in a control; wherein an increased levels of the at least one polypeptide of the EGF pathway in the sample as compared to the levels of the panel of polypeptides in the control identifies the mammalian tumor as treatable with a dual mTOR pathway inhibitor and EGF pathway inhibitor therapy.

57. The method of claim 54, wherein the at least one polypeptide of the EGF pathway comprises the phosphorylated ERK polypeptide.

58. The method of claim 57, wherein the detected pattern of expression, phosphorylation or both expression and phosphorylation of the phosphorylated ERK polypeptide is compared to a pattern of expression, phosphorylation or both expression and phosphorylation of the phosphorylated ERK polypeptide in a control; wherein an increased levels of the phosphorylated ERK polypeptide in the sample as compared to the levels the phosphorylated ERK polypeptide in the control identifies the mammalian tumor as treatable with a dual mTOR pathway inhibitor and EGF pathway inhibitor therapy.

59. The method of claim 54, wherein the at least one polypeptide of the EGF pathway comprises the phosphorylated MEK polypeptide.

60. The method of claim 59, wherein the detected pattern of expression, phosphorylation or both expression and phosphorylation of the phosphorylated MEK polypeptide is compared to a pattern of expression, phosphorylation or both expression and phosphorylation of the phosphorylated MEK polypeptide in a control; wherein an increased levels of the phosphorylated MEK polypeptide in the sample as compared to the levels the phosphorylated MEK polypeptide in the control identifies the mammalian tumor as treatable with a dual mTOR pathway inhibitor and EGF pathway inhibitor therapy.
